(19) United States
(12) Patent Application Publication
(10) Pub. No.: US 2023/0207090 A1
(43) Pub. Date: Jun. 29, 2023

(54) SYSTEMS, DEVICES, AND METHODS FOR DOSING PATTERN MANAGEMENT

(71) Applicant: ABBOTT DIABETES CARE INC., Alameda, CA (US)

(72) Inventors: Jonathan M. Fern, Alameda, CA (US); Diana W. Greenberg, San Francisco, CA (US); Panganamala Ashwin Kumar, Oakland, CA (US); Jessica Rose Floeh, Baltimore, MD (US); Lynne Lyons Carson, Burlingame, CA (US)

(21) Appl. No.: 17/925,692

(22) PCT Filed: May 27, 2021
(86) PCT No.: PCT/US2021/034510

§ 371 Date: Nov. 16, 2022

Related U.S. Application Data
(60) Provisional application No. 63/032,094, filed on May 29, 2020.

(51) Int. Cl. G16H 20/17

(52) U.S. Cl. CPC G16H 20/17

(57) ABSTRACT

Systems, devices, and methods for identifying and managing medication dosage patterns to assist in decisions for administration at, e.g., the time a meal is being consumed, are described. The application can include a new mealtime insulin dose-decisioning feature that is accessible through or in conjunction with an analyte monitoring application. Using retrospective analyte and medication dosing data only, the dosing pattern management application can display patterns in past dose administrations to facilitate easy and better dose decisions for diabetics on a regimen of multiple daily injections.

### CROSS-REFERENCE TO PATENT APPLICATION

The present application is a national phase entry under 35 U.S.C. § 371 of PCT Application No. PCT/US21/34510, filed May 27, 2021, which claims priority to U.S. Provisional Pat. Application No. 63/032,094, filed May 29, 2020, both of which are incorporated herein by reference in their entireties for all purposes.

### FIELD

The present subject matter broadly relates to systems, devices, and methods for the collection of information about analyte levels of certain individuals and information about medication doses that have been previously administered when the user was at or near a particular analyte level. The present subject matter further relates to processing, analyzing, and/or presenting this information for the purpose of correlating retrospective analyte and medication dosage information, identifying patterns in the retrospective analyte and medication dosage information, and presenting the patterns to the individual to assist in determining a treatment decision.

### BACKGROUND

The increased prevalence of Type-Two diabetes and metabolic syndrome over the past few decades has been attributed to changing diet and activity levels. For example, consumption of more readily available high glycemic index (GI) foods causes rapid post-prandial increase of blood glucose and insulin levels, which has a positive association with weight gain and obesity. These conditions can be further traced to an increased risk of developing these and other diseases.

The detection and/or monitoring of analyte levels, such as glucose, ketones, lactate, oxygen, hemoglobin A1C, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies or when additional glucose is needed to raise the level of glucose in their bodies.

Growing clinical data demonstrates a strong correlation between the frequency of glucose monitoring and glycemic control. Despite such correlation, many individuals diagnosed with a diabetic condition do not monitor their glucose levels as frequently as they should due to a combination of factors including inconvenience, testing discretion, pain associated with glucose testing and cost.

For example, for diabetics that require the administration of insulin, glucose levels are typically measured by performing a blood glucose measurement with a test strip or by using a glucose sensor inserted into the body. Maintaining multiple and separate devices, however, for purposes of monitoring analyte levels and administering medication can be burdensome to the patient. In addition, a lack of interoperability between different devices used by diabetics can create further inconvenience, e.g., where the medication delivery device, reader device and sensor device are each manufactured by a different party. For instance, requiring the patient to manually input information from one device to another can be cumbersome and prone to human error.

Current programs related to dosing often require the user to input meal or carbohydrate information regarding the meal that is about to be consumed. In addition to the fact that carbohydrate content is often difficult for the individual to determine accurately, such a requirement often discourages the user from using the program.

Thus, improved systems, devices, and methods for assisting individuals in making treatment decisions are needed.

### SUMMARY

Meal insulin dosing can be a big challenge, where the person is often relying on trial and error and spotty recollection. The person is often more comfortable adjusting insulin dose amounts rather than estimating meal size or carbohydrate contents of the meal. Most people taking insulin do not count carbohydrates. Moreover, hypoglycemia is a big concern. Thus, an application that is easy to use that may not require setup or participation by an HCP would be highly desirable over existing dosing applications. The application may present selective retrospective data such that it is easily actionable via a simple graphic approach.

The application may help users make sense of their insulin dosing patterns. The application may also provide guided interpretation of their insulin dosing history. The application my increase the user’s confidence in their insulin dosing decisions. The application may also assist the user in understanding the impact of their insulin doses on their glucose levels. The application may also assist a user to make decisions and determine actions in real-time or assist with correction dose decisions.

The dosing pattern management application may include a new mealtime insulin dose-decisioning feature that is accessible through or in conjunction with a glucose monitoring application or other diabetes-related application. Using retrospective glucose and insulin data only, the dosing pattern management application may display key data to facilitate easy and better dose decisions for diabetics (e.g., Type 2) on a regimen of multiple daily injections (MDI). The dosing pattern management application is intended to make it easier for diabetics on a regimen of MDIs to learn from the best decisions that they have made in the past to figure out which doses would be result in them remaining in the target glucose range. When users make a treatment decision that they are unsure of, the dosing pattern management application can help them make the dosage choice by showing the user which of their past doses were most effective in getting the user safely back to their goal range after meals.

There is a low likelihood of finding the exact same glucose and dose amount twice because of the nature of glucose monitoring combined with users’ adjustments of insulin dose amounts for various factors not necessarily glucose-related (i.e., more carbs, less carbs, sickness, etc.). In preferred embodiments at least, the dosing pattern management application solves this problem by grouping mealtime doses when the user’s glucose was around the same glucose. This allows the feature to still build a database of similar decisions even though there is not an exact match. Good past doses that resulted in the user reaching their target can be indicated as such to the user, for example in some embodiments they may be highlighted in green, and/or by any other indication, marker, icon, etc. Thus, a dose with more green tiles associated with it can show the user that that particular dose was successful in reaching the target multiple times. The dosing pattern management application may also warn the user which doses resulted in the user having a hypoglycemic episode (e.g., below 70 mg/dL) in the four hours after the dose administration.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be depicted schematically rather than literally or precisely.

FIG. 1 is a high level diagram depicting an example embodiment of an analyte monitoring system for real time analyte (e.g., glucose) measurement, data acquisition and/or processing.

FIG. 2A is a block diagram depicting an example embodiment of a reader device configured as a smartphone.

FIG. 2B is a block diagram depicting an example embodiment of a sensor control device.

FIG. 3A depicts components used with an embodiment of a dosing pattern management application.

FIGS. 3B-3C are flow diagrams depicting an example embodiment of a method for managing dosing patterns.

FIG. 4A depicts example embodiments of graphical user interfaces that show a dose of a medication has been administered.

FIG. 4B depicts an example embodiment of a graphical user interface that the user can use to edit information regarding the medication that was administered.

FIGS. 5A-5B depict example embodiments of graphical user interfaces where the user can select their goal.

FIG. 6A depicts an example embodiment of a graphical user interface where the user can access the dosing pattern management application from an analyte monitoring application.

FIGS. 6B-6D depict example embodiments of graphical user interfaces that show past doses associated with a particular analyte level.

FIGS. 7A-7D depict example embodiments of graphical user interfaces that show additional dosing information associated with specific doses.

FIGS. 8A-8B depict example embodiments of graphical user interfaces that show messages that can prompt a user to check their past dosages.

FIG. 9A depicts an example embodiment of a graphical user interface where the user can access another embodiment of the dosing pattern management application from an analyte monitoring application.

FIGS. 9B-9C depict example embodiments of graphical user interfaces that show dosages with corresponding star ratings.

FIGS. 10A-10B depict additional example embodiments of graphical user interfaces that show dosages with corresponding star ratings.

FIGS. 11A-11B depict example embodiments of alternative graphical user interfaces that allow the user to add tags to doses.

FIG. 12A depicts an example embodiment of a graphical user interface where the user can access another embodiment of the dosing pattern management application from an analyte monitoring application.

FIG. 12B depicts an example embodiment of a graphical user interfaces that shows dosages with tags.

FIGS. 13A-13B depict additional example embodiments of alternative graphical user interfaces that display the doses according to associated tags.

FIGS. 14A-14K-1depict various embodiments of the methods, systems, and interfaces described herein.

FIGS. 15A-15N depict various embodiments of the methods, systems, and interfaces described herein.

FIGS. 16A-16J depict various embodiments of the methods, systems, and interfaces described herein.

FIGS. 17A-17C depict various graphical representations for evaluating the effectiveness of doses.

FIG. 18 depicts an example embodiment of a graphical user interface that shows a suggested dose.

FIG. 19A depicts an example embodiment of a graphical user interface that shows a range of doses.

FIGS. 19B-19D depict example embodiments of graphical depictions of various past doses.

FIGS. 20A-20D depict example embodiments of graphical user interfaces or reports that show how many injections or meals have met or almost met a target.

FIGS. 21A-21C graphical user interfaces or reports that show how many meals have met a target in a time period.

### DETAILED DESCRIPTION

Provided herein are example embodiments of systems, devices, and methods for managing dosing patterns. For example, when a user is trying to determine an amount of insulin to inject before consuming a meal, an application can use retrospective glucose and insulin data to assist the user in making an appropriate dosage determination. Based on the user’s current glucose level, the application can display previous doses of insulin that the user administered when he had an identical or similar glucose level. Moreover, the application can display an indication of whether a particular dose resulted in the user staying in a goal or target range or if the user experienced hypoglycemia after administration.

Before describing this subject matter in greater detail, it is worthwhile to describe example embodiments of systems, devices, and methods with which the subject matter can be implemented.

A number of systems have been developed for the automatic monitoring of the analyte(s), like glucose, in bodily fluid such as in the blood stream, in interstitial fluid (“ISF”), dermal fluid of the dermal layer, or in other biological fluid. Some of these systems are configured so that at least a portion of a sensor is positioned below a skin surface of a user, e.g., in a blood vessel or in the subcutaneous tissue of a user, to obtain information about at least one analyte of the body.

As such, these systems can be referred to as “in vivo” monitoring systems. In vivo analyte monitoring systems include “Continuous Analyte Monitoring” systems (or “Continuous Glucose Monitoring” systems) that can broadcast data from a sensor control device to a reader device continuously without prompting, e.g., automatically according to a broadcast schedule. In vivo analyte monitoring systems also include “Flash Analyte Monitoring” systems (or “Flash Glucose Monitoring” systems or simply “Flash” systems) that can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

The in vivo analyte monitoring systems can be differentiated from “in vitro” systems that contact a biological sample outside of the body (or rather “ex vivo”) and that typically include a meter device that has a port for receiving an analyte test strip carrying bodily fluid of the user, which can be analyzed to determine the user’s blood sugar level. While in many of the present embodiments the monitoring is accomplished in vivo, the embodiments disclosed herein can be used with in vivo analyte monitoring systems that incorporate in vitro capability, as well has purely in vitro or ex vivo analyte monitoring systems.

The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “reader device” (or simply a “reader”), “handheld electronics” (or a handheld), a “portable data processing” device or unit, a “data receiver,” a “receiver” device or unit (or simply a receiver), or a “remote” device or unit, to name a few. Other devices such as personal computers have also been utilized with or incorporated into in vivo and in vitro monitoring systems.

### Embodiments of In Vivo Monitoring Systems

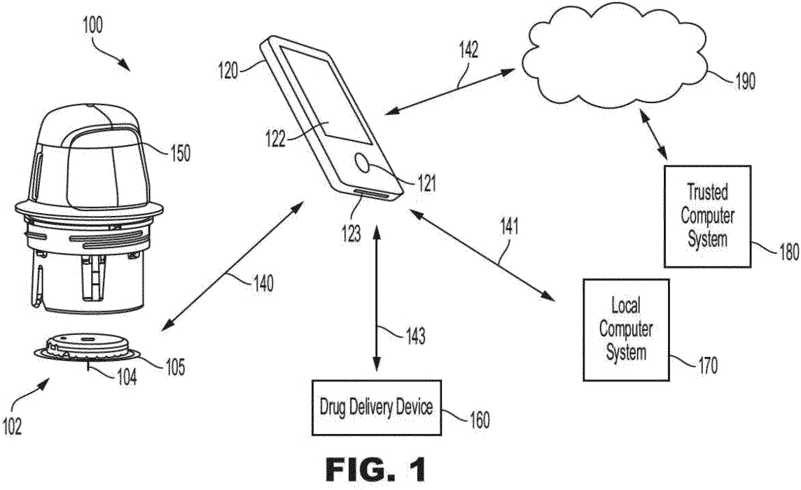

For purpose of illustration, and not limitation, the graphical user interfaces and associated software described herein may be used in connection with an exemplary analyte monitoring system as depicted in FIG. 1. FIG. 1 is an illustrative view depicting an example in vivo analyte monitoring system 100 with which any and/or all of the embodiments described herein can be used. System 100 can have a sensor control device 102 and a reader device 120 that communicate with each other over a local communication path (or link) 140, which can be wired or wireless, and uni-directional or bi-directional. In embodiments where local communication path 140 is wireless, any near field communication (NFC®) protocol, RFID® protocol, BLUETOOTH® or BLUETOOTH® Low Energy protocol, WI-FI® protocol, proprietary protocol, or the like can be used, including those communication protocols in existence as of the date of this filing or their later developed variants.

BLUETOOTH® is a well-known standardized short range wireless communication protocol, and BLUETOOTH® Low Energy is a version of the same that requires less power to operate. BLUETOOTH® Low Energy (BLUETOOTH® LE, BTLE™, BLE™) is also referred to as BLUETOOTH SMART® or BLUETOOTH SMART READY®. A version of BTLE™ is described in the BLUETOOTH® Specification, version 4.0, published Jun. 30, 2010, which is explicitly incorporated by reference herein for all purposes. The term “NFC®” applies to a number of protocols (or standards) that set forth operating parameters, modulation schemes, coding, transfer speeds, frame format, and command definitions for NFC® devices. The following is a non-exhaustive list of examples of these protocols, each of which (along with all of its sub-parts) is incorporated by reference herein in its entirety for all purposes: ECMA-340, ECMA-352, ISO/IEC 14443, ISO/IEC 15693, ISO/IEC 16000-3, ISO/IEC 18092, and ISO/IEC 21481.

Reader device 120 is also capable of wired, wireless, or combined communication, either bidirectional or unidirectional, with either or all of: an drug delivery device 160 (such as a connected insulin pen) over communication path (or link) 143, a local computer system 170 over communication path (or link) 141, and with a network 190 over communication path (or link) 142. The same wireless protocols described for link 140 can likewise be used for all or part of links 141, 142, and 143.

Reader device 120 can communicate with any number of entities through network 190, which can be part of a telecommunications network, such as a WI-FI® network, a local area network (LAN), a wide area network (WAN), the internet, or other data network for uni-directional or bi-directional communication. A trusted computer system 180 can be accessed through network 190. In an alternative embodiment, communication paths 141 and 142 can be the same path which can include the network 190 and/or additional networks. All communications over paths 140, 141, 142, and 143 can be encrypted and sensor control device 102, reader device 120, drug delivery device 160, remote computer system 170, and trusted computer system 180 can each be configured to encrypt and decrypt those communications sent and received.

Variants of devices 102 and 120, as well as other components of an in vivo-based analyte monitoring system that are suitable for use with the system, device, and method embodiments set forth herein, are described in U.S. Pat. Publication No. 2011/0213225 (the ‘225 Publication), which is incorporated by reference herein in its entirety for all purposes. Variations of devices 102 and 120 including connected drug delivery devices 160, such as a connected insulin pen, that are suitable for use with the system, device, and method embodiments set forth herein, are described in WO 2018/152241, which is incorporated by reference herein in its entirety for all purposes.

Sensor control device 102 can include a housing 103 containing in vivo analyte monitoring circuitry and a power source (not shown). The in vivo analyte monitoring circuitry can be electrically coupled with an analyte sensor 104 that can extend through an adhesive patch 105 and project away from housing 103. Adhesive patch 105 contains an adhesive layer (not shown) for attachment to a skin surface of the body of the user. Other forms of body attachment to the body may be used, in addition to or instead of adhesive.

Sensor 104 is adapted to be at least partially inserted into the body of the user, where it can make fluid contact with that user’s body fluid (e.g., interstitial fluid (ISF), dermal fluid, or blood) and be used, along with the in vivo analyte monitoring circuitry, to measure analyte-related data of the user. Generally, sensor control device 102 and its components can be applied to the body with a mechanical applicator 150 in one or more steps, as described in the incorporated ‘225 Publication, or in any other desired manner.

After activation, sensor control device 102 can wirelessly communicate the collected analyte data (such as, for example, data corresponding to monitored analyte level and/or monitored temperature data, and/or stored historical analyte related data) to reader device 120 where, in certain embodiments, it can be algorithmically processed into data representative of the analyte level of the user and then displayed to the user and/or otherwise incorporated into a diabetes monitoring regime.

Various embodiments disclosed herein relate to reader device 120, which can have a user interface including one or more of a display 122, keyboard, optional user interface component 121, and the like. Here, display 122 can output information to the user and/or accept an input from the user (e.g., if configured as a touch screen). Reader device 120 can include one or more optional user interface components 121, such as a button, actuator, touch sensitive switch, capacitive switch, pressure sensitive switch, jog wheel or the like. Reader device 120 can also include one or more data communication ports 123 for wired data communication with external devices such as local computer system 170. Reader device 120 may also include an integrated or attachable in vitro meter, including an in vitro test strip port (not shown) to receive an in vitro analyte test strip for performing in vitro blood analyte measurements.

Drug delivery device 160 is capable of injecting or infusing a drug, such as but not limited to insulin, into the body of the individual wearing sensor control device 102. Like reader device 120, the drug delivery device can include processing circuitry, non-transitory memory containing instructions executable by the processing circuitry, wireless or wired communication circuitry, and a user interface including one or more of a display, touchscreen, keyboard, an input button or instrument, and the like. Drug delivery device 160 can include a connected insulin pen, a drug reservoir, a pump, an infusion tube, and an infusion cannula configured for at least partial implantation into the user’s body. The pump can deliver insulin from the reservoir, through the tube, and then through the cannula into the user’s body. Drug delivery device 160 can include instructions, executable by the processor, to control the pump and the amount of insulin delivered. These instructions can also cause calculation of insulin delivery amounts and durations (e.g., a bolus infusion and/or a basal infusion profile) based on analyte level measurements obtained directly or indirectly from sensor control device 102. Alternatively, calculations of insulin delivery amounts and durations, and the control of the pump, can be performed by reader device 120 directly. The drug delivery device can be configured to communicate directly with reader device 120 in the form of a closed loop or semi-closed loop system. Alternatively, the drug delivery device can include the functionality of reader device 120 described herein, or vice versa, to arrive at one integrated reader and drug delivery device.

Computer system 170 may be a personal or laptop computer, a tablet, or other suitable data processing device. Computer 170 can be either local (e.g., accessible via a direct wired connection such as USB) or remote to reader device 120 and can be (or include) software for data management and analysis and communication with the components in analyte monitoring system 100. Operation and use of computer 170 is further described in the ‘225 Publication incorporated herein by reference. Analyte monitoring system 100 can also be configured to operate with a data processing module (not shown), also as described in the incorporated ‘225 Publication.

Trusted computer system 180 can be used to perform authentication of sensor control device 102 and/or reader device 120, used to store confidential data received from devices 102 and/or 120, used to output confidential data to devices 102 and/or 120, or otherwise configured. Trusted computer system 180 can include one or more computers, servers, networks, databases, and the like. Trusted computer system 180 can be within the possession of the manufacturer or distributor of sensor control device 102, either physically or virtually through a secured connection, or can be maintained and operated by a different party (e.g., a third party).

Trusted computer system 180 can be trusted in the sense that system 100 can assume that computer system 180 provides authentic data or information. Trusted computer system 180 can be trusted simply by virtue of it being within the possession or control of the manufacturer, e.g., like a typical web server. Alternatively, trusted computer system 180 can be implemented in a more secure fashion such as by requiring additional password, encryption, firewall, or other internet access security enhancements that further guard against counterfeiter attacks or attacks by computer hackers.

The processing of data and the execution of software within system 100 can be performed by one or more processors of reader device 120, computer system 170, and/or sensor control device 102. For example, raw data measured by sensor 104 can be algorithmically processed into a value that represents the analyte level and that is readily suitable for display to the user, and this can occur in sensor control device 102, reader device 120, or computer system 170. This and any other information derived from the raw data can be displayed in any of the manners described above (with respect to display 122) on any display residing on any of sensor control device 102, reader device 120, or computer system 170. The information may be utilized by the user to determine any necessary corrective actions to ensure the analyte level remains within an acceptable and/or clinically safe range.

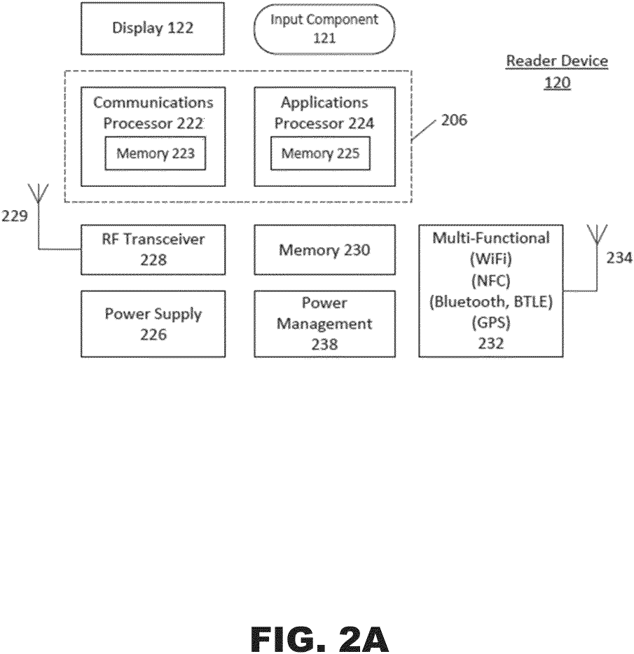

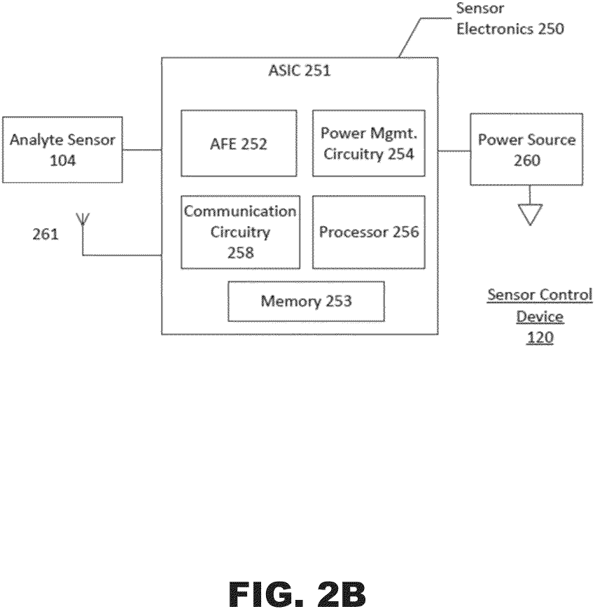

FIGS. 2A-2B depict example embodiments of reader device 120 and sensor control device 102, respectively. As discussed above, reader device 120 can be a mobile communication device such as, for example, a WI-FI® or internet enabled smartphone, tablet, or personal digital assistant (PDA). Examples of smartphones can include, but are not limited to, those phones based on a WINDOWS® operating system, ANDROID® operating system, IPHONE® operating system, PALM WEBOS™, BLACKBERRY® operating system, or SYMBIAN™ operating system, with network connectivity for data communication over the internet or a local area network (LAN).

Sensor control device 102 and reader device can communicate with integrated drug delivery device 160 via communication path 143 using a wired or wireless technique (or combinations thereof). Communication across communication path can be direct from sensor control device 102 to integrated device 160 without an intermediary. In alternative embodiments, sensor control device 102 can communicate to integrated drug delivery device 160 indirectly through an intermediary, e.g., by communicating to a first device that then communicates to integrated device 160. That first device can be, e.g., a display device or data processing module as described in U.S. Pat. Publication No. 2011/0213225 (the ‘225 Publication), which is incorporated by reference herein in its entirety for all purposes.

Reader device 120 can also be configured as a mobile smart wearable electronics assembly, such as an optical assembly that is worn over or adjacent to the user’s eye (e.g., a smart glass or smart glasses, such as GOOGLE GLASS™). This optical assembly can have a transparent display that displays information about the user’s analyte level (as described herein) to the user while at the same time allowing the user to see through the display such that the user’s overall vision is minimally obstructed. The optical assembly may be capable of wireless communications similar to a smartphone. Other examples of wearable electronics include devices that are worn around or in the proximity of the user’s wrist (e.g., a watch, etc.), neck (e.g., a necklace, etc.), head (e.g., a headband, hat, etc.), chest, or the like.

FIG. 2A is a block diagram of an example embodiment of a reader device 120 according to various embodiments disclosed herein. In this example, the reader device 120 is in the form of a smartphone, upon which the various software, applications, and graphical user interfaces disclosed herein can reside. Here, reader device 120 includes an input component 121, display 122, and processing hardware 206, which can include one or more processors, microprocessors, controllers, and/or microcontrollers, each of which can be a discrete chip or distributed amongst (and a portion of) a number of different chips. Here, processing hardware 206 includes a communications processor 222 having on-board non-transitory memory 223 and an applications processor 224 having on-board non-transitory memory 225. Reader device 120 further includes an RF transceiver 228 coupled with an RF antenna 229, a memory 230, multi-functional circuitry 232 with one or more associated antennas 234, a power supply 226, and power management circuitry 238. FIG. 2A is an abbreviated representation of the internal components of a smartphone, and other hardware and functionality (e.g., codecs, drivers, glue logic, etc.) can of course be included.

Communications processor 222 can interface with RF transceiver 228 and perform analog-to-digital conversions, encoding and decoding, digital signal processing and other functions that facilitate the conversion of voice, video, and data signals into a format (e.g., in-phase and quadrature) suitable for provision to RF transceiver 228, which can then transmit the signals wirelessly. Communications processor 222 can also interface with RF transceiver 228 to perform the reverse functions necessary to receive a wireless transmission and convert it into digital data, voice, and video.

Applications processor 224 can be adapted to execute the operating system and any software applications that reside on reader device 120 (such as any sensor interface application or analyte monitoring application that includes, e.g., SLL 304), process video and graphics, and perform those other functions not related to the processing of communications transmitted and received over RF antenna 229. Any number of applications can be running on reader device 120 at any one time, and will typically include one or more applications that are related to a diabetes monitoring regime, in addition to the other commonly used applications that are unrelated to such a regime, e.g., email, calendar, weather, etc.

Memory 230 can be shared by one or more of the various functional units present within reader device 120, or can be distributed amongst two or more of them (e.g., as separate memories present within different chips). Memory 230 can also be a separate chip of its own. Memory 230 is non-transitory, and can be volatile (e.g., RAM, etc.) and/or non-volatile memory (e.g., ROM, flash memory, F-RAM, etc.).

Multi-functional circuitry 232 can be implemented as one or more chips and/or components, including communication circuitry, that perform other functions such as local wireless communications (e.g., WI-FI®, BLUETOOTH®, BLUETOOTH® Low Energy) and determining the geographic position of reader device 120 (e.g., global positioning system (GPS) hardware). One or more other antennas 234 are associated with the functional circuitry 232 as needed.

Power supply 226 can include one or more batteries, which can be rechargeable or single-use disposable batteries. Power management circuitry 238 can regulate battery charging and power supply monitoring, boost power, perform DC conversions, and the like. As mentioned, reader device 120 may also include one or more data communication ports such as USB port (or connector) or RS-232 port (or any other wired communication ports) for data communication with a remote computer system 170 (see FIG. 1), or sensor control device 102, to name a few.

FIG. 2B is a block schematic diagram depicting an example embodiment of sensor control device 102 having analyte sensor 104 and sensor electronics 250 (including analyte monitoring circuitry). Although any number of chips can be used, here the majority of the sensor electronics 250 are incorporated on a single semiconductor chip 251 that can be, e.g., a custom application specific integrated circuit (ASIC). Shown within ASIC 251 are several high-level functional units, including an analog front end (AFE) 252, power management circuitry 254, processor 256, and communication circuitry 258 (which can be implemented as a transmitter, receiver, transceiver, passive circuit, or otherwise according to the communication protocol). In this embodiment shown in FIG. 2B, both AFE 252 and processor 256 are used as analyte monitoring circuitry, but in other embodiments either circuit can perform the analyte monitoring function. Processor 256 can include one or more processors, microprocessors, controllers, and/or microcontrollers.

A non-transitory memory 253 is also included within ASIC 251 and can be shared by the various functional units present within ASIC 251, or can be distributed amongst two or more of them. Memory 253 can be volatile and/or non-volatile memory. In this embodiment, ASIC 251 is coupled with power source 260, which can be a coin cell battery, or the like. AFE 252 interfaces with in vivo analyte sensor 104 and receives measurement data therefrom and outputs the data to processor 256 in digital form, which in turn processes the data to arrive at the end-result analyte discrete and trend values, etc. This data can then be provided to communication circuitry 258 for sending, by way of antenna 261, to reader device 120 (not shown) where further processing can be performed by, e.g., the sensor interface application. It should be noted that the functional components of ASIC 251 can also be distributed amongst two or more discrete semiconductor chips.

Performance of the data processing functions within the electronics of the sensor control device 102 provides the flexibility for system 100 to schedule communication from sensor control device 102 to reader device 120, which in turn limits the number of unnecessary communications and can provide further power savings at sensor control device 102.

Information may be communicated from sensor control device 102 to reader device 120 automatically and/or continuously when the analyte information is available, or may not be communicated automatically and/or continuously, but rather stored or logged in a memory of sensor control device 102, e.g., for later output.

Data can be sent from sensor control device 102 to reader device 120 at the initiative of either sensor control device 102 or reader device 120. For example, in many example embodiments sensor control device 102 can communicate data periodically in an unprompted or broadcast-type fashion, such that an eligible reader device 120, if in range and in a listening state, can receive the communicated data (e.g., sensed analyte data). This is at the initiative of sensor control device 102 because reader device 120 does not have to send a request or other transmission that first prompts sensor control device 102 to communicate. Broadcasts can be performed, for example, using an active WI-FI®, BLUETOOTH®, or BTLE® connection. The broadcasts can occur according to a schedule that is programmed within device 102 (e.g., about every 1 minute, about every 5 minutes, about every 10 minutes, or the like). Broadcasts can also occur in a random or pseudorandom fashion, such as whenever sensor control device 102 detects a change in the sensed analyte data. Further, broadcasts can occur in a repeated fashion regardless of whether each broadcast is actually received by a reader device 120.

System 100 can also be configured such that reader device 120 sends a transmission that prompts sensor control device 102 to communicate its data to reader device 120. This is generally referred to as “on-demand” data transfer. An on-demand data transfer can be initiated based on a schedule stored in the memory of reader device 120, or at the behest of the user via a user interface of reader device 120. For example, if the user wants to check his or her analyte level, the user could perform a scan of sensor control device 102 using an NFC®, BLUETOOTH®, BTLE®, or WI-FI® connection. Data exchange can be accomplished using broadcasts only, on-demand transfers only, or any combination thereof.

Accordingly, once a sensor control device 102 is placed on the body so that at least a portion of sensor 104 is in contact with the bodily fluid and electrically coupled to the electronics within device 102, sensor derived analyte information may be communicated in on-demand or unprompted (broadcast) fashion from the sensor control device 102 to a reader device 120. On-demand transfer can occur by first powering on reader device 120 (or it may be continually powered) and executing a software algorithm stored in and accessed from a memory of reader device 120 to generate one or more requests, commands, control signals, or data packets to send to sensor control device 102. The software algorithm executed under, for example, the control of processing hardware 206 of reader device 120 may include routines to detect the position of the sensor control device 102 relative to reader device 120 to initiate the transmission of the generated request command, control signal and/or data packet.

Analyte level data can be transferred from system 100 to a dosing pattern management application.

### Dosing Pattern Management Application

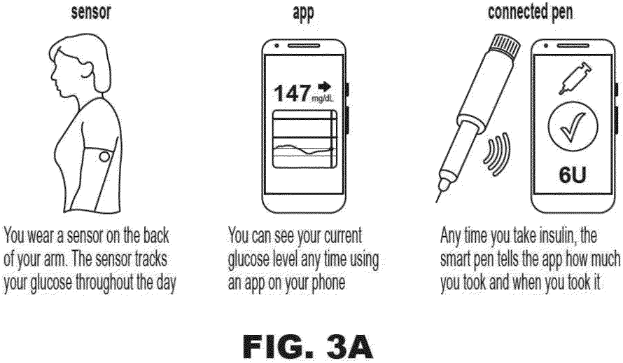

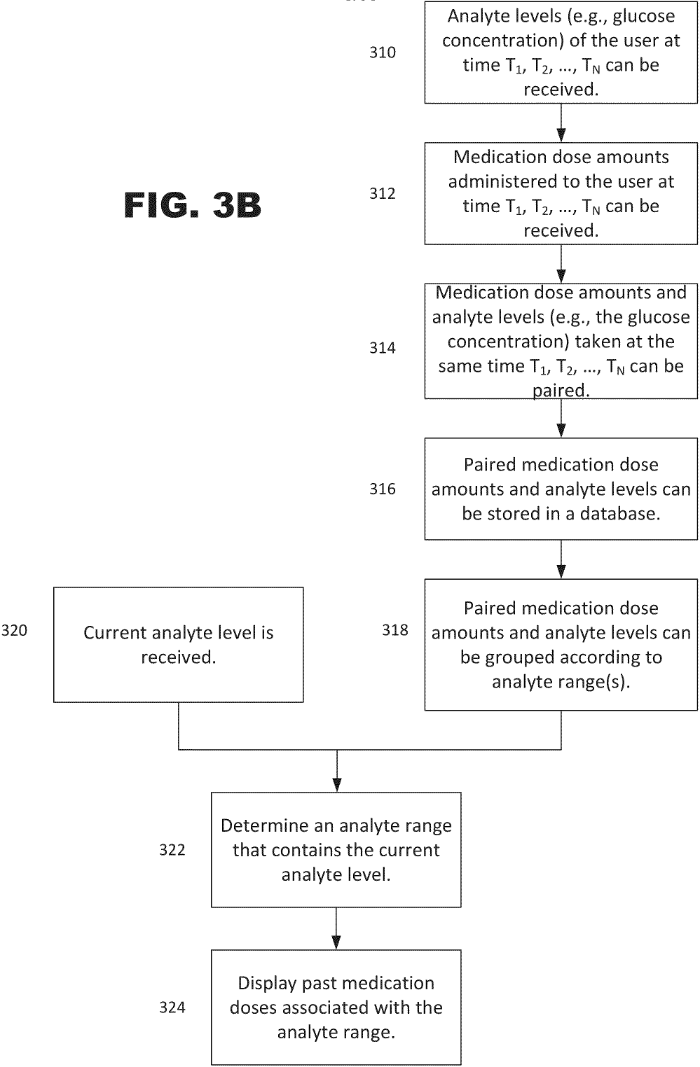

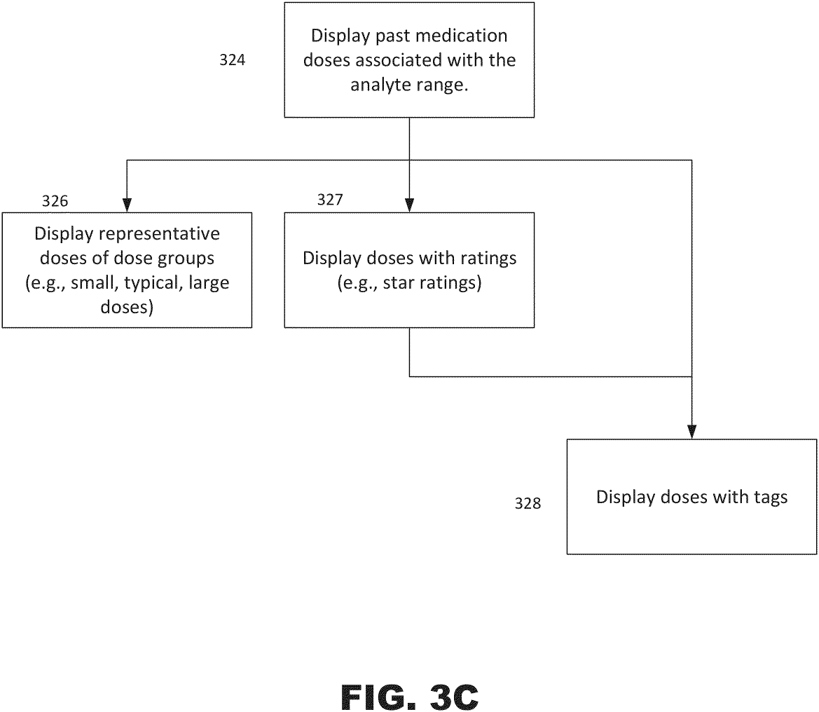

The dosing pattern management application 300 can provide insights to the user as to what previous doses were administered when the user was at or near a particular analyte level or analyte range. The dosing management application 300 may be a stand-alone application or may be incorporated into another application or software in part or in whole. As seen in FIG. 3A, the system includes the dosing pattern management application 300 downloaded and installed on an electronic device 120 that is in communication with a sensor control device 102 and a medication delivery device 160, such as a connected insulin pen. The dosing pattern management application 300 can receive analyte levels from the sensor control device 102, a sensor or user interface application, or other network server, up to a cloud and transferred from the cloud to the dosing pattern management application. For example, analyte data can be uploaded to a first server or group of servers responsible for collecting analyte data, and then downloaded to the dosing pattern management application by a second server or group of servers responsible for downloading the data for use by the dosing pattern management application.

The dosing pattern management application 300 can similarly receive data regarding dosages administered at different times to the individual through the medication delivery device 160 (e.g., an insulin pen or an insulin pump). Each time a user injects an insulin dose using their connected medication delivery device, the medication dose amount and the analyte level at the time of (or near the time of) the injection can be paired. This pairing can represent a mealtime dose decision that was made by the user. Pairing the dose and the analyte level at the time of dosing is important because users make dose decisions based on both the starting analyte level and the food about to be consumed.

The dosing pattern management application 300 can associate the data regarding each of the previously administered doses with the corresponding analyte level (e.g., glucose level) that was measured in the individual at or near the time of administration of the medication, e.g., insulin. The analyte level may have been measured at the same time that the dose was administered, or alternatively within about 1 minute, alternatively within about 2 minutes, alternatively within about 3 minutes, alternatively within about 4 minutes, alternatively within about 5 minutes, alternatively within about 10 minutes, alternatively within about 15 minutes, alternatively within about 20 minutes of the time of administration.

The administered doses can be grouped according to which doses were administered for a corresponding analyte range that contains the corresponding analyte level, rather than only reporting doses administered for the identical analyte level. For example, if the measured glucose level is 220 mg/dL, the program may identify any dosages that were administered when the individual had a measured analyte level of between about 170 mg/dL to 270 mg/dL (± 50 mg/dL), or between about 180 mg/dL to 260 mg/dL (± 40 mg/dL), or between about 190 mg/dL to 250 mg/dL (± 30 mg/dL) or between about 200 mg/dL to 240 mg/dL (± 20 mg/dL), or between about 210 mg/dL to 230 mg/dL (± 10 mg/dL), or combinations thereof. The analyte range can be defined by an analyte level ± 30% of the analyte level, alternatively an analyte level ± 25% of the analyte level, an analyte level ± 20% of the analyte level, an analyte level ± 15% of the analyte level, or an analyte level ± 10% of the analyte level.

The analyte range may change over time. For example, in the first 2 weeks of use prior to there being enough paired data in the app to surface patterns, the user data for each analyte range may be quite sparse while the application is recording data. Thus, the application can use a wider range (i.e. ±50 mg/dL) during, e.g., the first 2-3 weeks of use. As the dosing pattern management application 300 continues to collect data and data becomes more abundant, the application 300 can continue to tighten the analyte range used for the feature (e.g., ±40 mg/dL, then ±30 mg/dL, then ±20 mg/dL) such that the analyte range will be smaller. The boundaries of the analyte range are determined based on the availability of data and may not be user-settable.

FIG. 3B is a flowchart diagram showing steps of an example method for managing dose patterns. In step 310, analyte levels (e.g., glucose concentrations or levels) of the user at various times (T₁, T₂, ..., T_{N}) can be received by the application 300. At step 312, insulin dose amounts that were administered to the user at various times (T₁, T₂, ..., T_{N}) can be received by the application 300. In step 312, the medication dose amounts, and the analyte levels taken at the same or similar times (T₁, T₂, ..., T_{N}) can be paired. In step 316, the paired medication dose amounts and analyte levels can be stored in a database. In step 318, the paired medication dose amounts and analyte levels can be grouped according to analyte range(s). In step 320, the current analyte level of the user is received by the application. In step 322, an analyte range that contains the current analyte level is determined. In step 324, the application displays past medication doses associated with the analyte range.

FIG. 3C is a continuation of the flowchart diagram of FIG. 3B and outlines various ways in which the past medication doses can be outputted and displayed. At step 326, the past medication doses can be divided into multiple (e.g., 3) groups and a representative dose of each group could be displayed. In another embodiment, at step 327, the past medication doses could be displayed with a rating (e.g., a star rating). In another embodiment, at step 328, the past medication doses could be displayed with tags, alone or in addition to the star ratings.

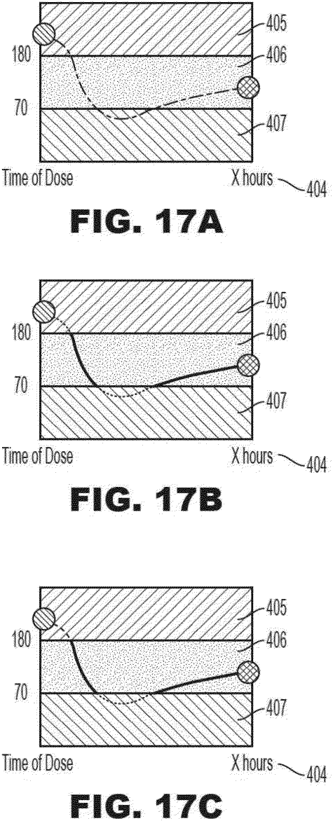

Past doses can be displayed in different ways to quickly show the reader that particular dose was good (e.g., resulted in the user reaching their goal range in a predetermined time after administration), ineffective (e.g., did not result in the user reaching their goal range in a predetermined time after administration), or bad (e.g., resulted in hypoglycemia in the user in a predetermined time after administration). Doses can be evaluated in different ways to determine if the dose is good, effective, or better than another dose. To determine if a particular dose was good or ineffective, the application may analyze the measured analyte level (e.g., glucose level) of the user for a predetermined time (e.g., about 1 hour, alternatively about 2 hours, alternatively about 3 hours, alternatively about 4 hours) after administration to see if the measured analyte level reached the user’s goal range. As seen in FIG. 17A, a blood glucose endpoint after a certain time period 404 (e.g., 1 hour, alternatively 2 hours, alternatively 3 hours, alternatively 4 hours) can be used. If the user’s blood glucose level was within a target range 406 (e.g., between about 70 mg/dL and about 180 mg/dL) at the end of the certain time period 404, then the dose may be classified as a “good” dose. As seen in FIG. 17B, a total time in a target range 406 during a time period 404 after the dose can also be used to determine if a dose is good or effective. The total time in the target range 406 can be an amount of time (e.g., number of hours) or a percentage of a total amount of time in a target range. As seen in FIG. 17C, a percentage of time in each range during a time period after the dose can be used to determine if a dose is good or effective. The different ranges may be in a target range 406 (e.g., between about 70 mg/dL and about 180 mg/dL), below a target range 407 (e.g., below about 70 mg/dL), and above a target range 405 (e.g., above about 180 mg/dL). The amounts of time in the various ranges may be weighted and the doses may be ranked by a weighted proportion.

To determine if a particular dose is bad because it resulted in a period of hypoglycemia in the user, the application may analyte the measured analyte data to determine if the measured analyte level fell below a hypoglycemia threshold. The hypoglycemia threshold may be set by the application or may be set by the user at, e.g., below 80 mg/dL, alternatively below 75 mg/dL, alternatively below 70 mg/dL, alternatively below 65 mg/dL.

### Dose Groups

The application 300 may analyze the doses associated with a particular analyte range and identify the smallest dose, the largest dose, the average dose, the median dose, or the dose given most frequently (the mode dose). Furthermore, the application can display the smallest, largest, and typical (e.g., median or dose given most frequently) doses so that the user can see the range and variety of the past doses administered under similar circumstances (e.g., similar analyte levels).

The application 300 may also divide the doses administered for a given analyte range into multiple groups, e.g., 2, 3, 4, or 5 groups, based on the amount of the medication in each dose. For example, the doses may be ordered according to the amount of medication in each dose, e.g., smallest doses to largest doses or largest doses to smallest doses. The doses may then be divided into 3 groups, with the small, medium (or typical), and large dose amounts grouped together. In other words, the first (e.g., small or smallest) dose group could contain the smallest dose administered, the second (e.g., large or largest) dose group could contain the largest dose administered, and the third (medium or typical) group could contain the median dose administered. Within each group, a representative dose may be displayed. The representative dose may be the mode dose (i.e., dose that occurs most frequently within that group). Alternatively, the minimum or maximum dose of a particular group may be displayed. For example, the smallest dose administered of the first group may be displayed and the largest dose of the second group may be displayed.

The display can also include an indication of when (e.g., time and/or date) these doses were administered and may also include a visual indication (e.g., date entry tile is colored green 376 or other positive indication) if the administered dose resulted in the user achieving a goal range. If the dose did not bring the user within the goal range (e.g., the user’s glucose level remained above the goal range), the date entry tile for that dose may be colored gray 378. If, however, the dose resulted in hypoglycemia in the user (e.g., below 70 mg/dL), then the dose tile may be colored gray 378 and also include a hypoglycemia marker 380 (e.g., a sad face emoji or an emoji showing typical hypoglycemia symptoms such as shakiness and sweating (e.g., an emoji with a sweat band). If the user achieved their goal range, but the user had any hypoglycemia within about 30 minutes to about 4 hours after the injection, the tile can remain gray because the occurrence of hypoglycemia trumps the good dose decision.

The application can also group the doses according to a time period in which the doses were administered or according to a meal with which they were administered. For instance, the doses may be associated with breakfast, lunch, dinner, or snacks. The application may also analyze and group correction doses, which are not associated with any meal, together. Alternatively, the doses may be associated with morning, afternoon, evening, and overnight. If the dose is not associated with the correct meal, the user can override a default meal setting and correct the type of meal associated with a particular dose. The application may also allow the users to set typical times for breakfast, lunch, and dinner. Such a feature can be helpful where the user has an eating schedule that differs from a typical meal schedule. The application may let the user filter which meal or time period to view. Each meal or time period may display a variety of doses (e.g., “smallest,” “typical,” “largest”).

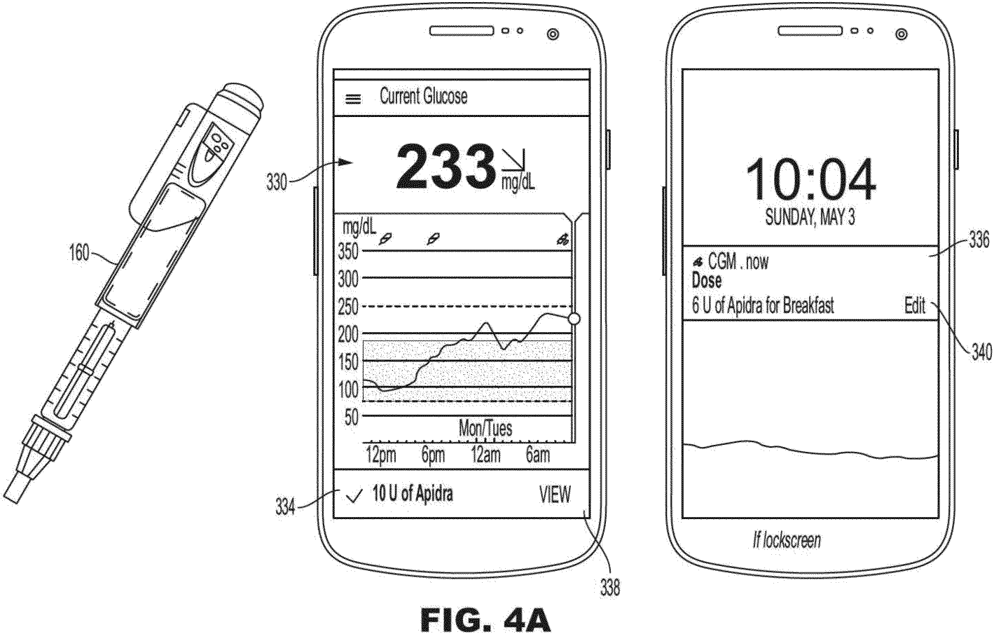

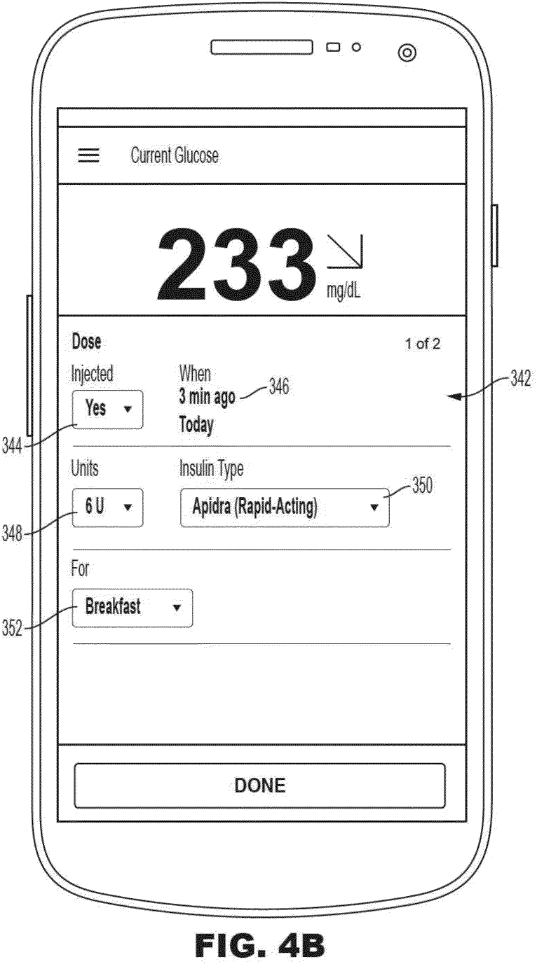

As seen in FIG. 4A, with medication delivery device 160 connected to the dosing pattern management application 300 and a sensor user interface application 330, a window 334 or alert 334 can appear to indicate that a dose was just administered. The window 334 or alert 336 may indicate the type of medication administered, the amount, and the meal with which the dose is associated. Clicking on or selecting a view 338 or edit 340 link, or simply tapping on the notification can open up a dose details screen 342 (see FIG. 4B), in which the user can edit various fields including method of administration (Injected - Yes/No) 344, time of administration 346, amount of medication administered 348, type of medication 350, and the corresponding meal with which the medication was administered 352.

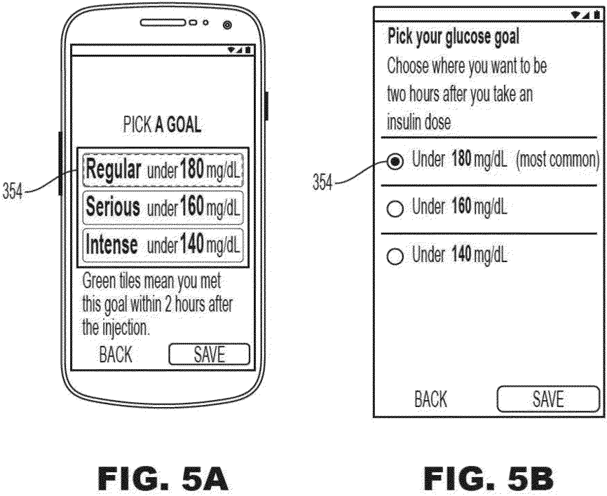

The user can also select a goal range 354 with which to compare the resulting analyte levels after a medication dose was administered by setting a maximum analyte level of the goal range or target range as seen in FIGS. 5A-5B. For example, the user can select under 180 mg/dL, alternatively under 170 mg/dL, alternatively under 160 mg/dL, alternatively under 150 mg/dL, alternatively under 140 mg/dL, alternatively under 130 mg/dL, and/or alternatively under 120 mg/dL. Moreover, the user may be able to enter a goal range rather than select preexisting choices (not shown). The goal range may have a minimum setting of under 100 mg/dL and a maximum setting of under 200 mg/dL.

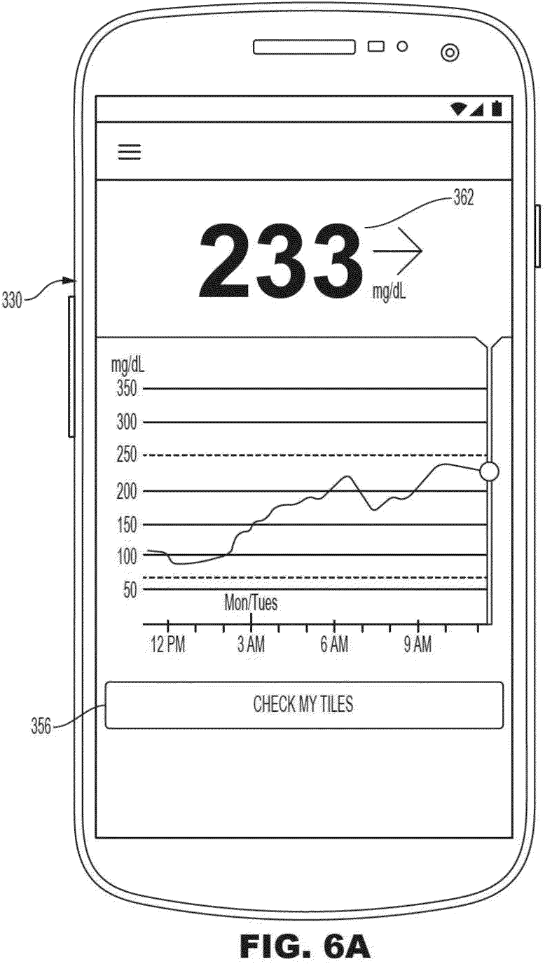

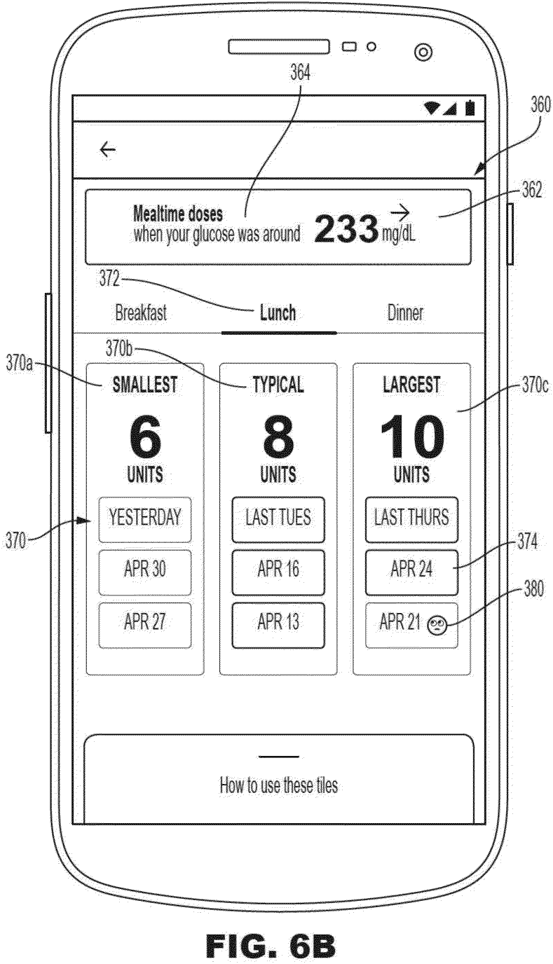

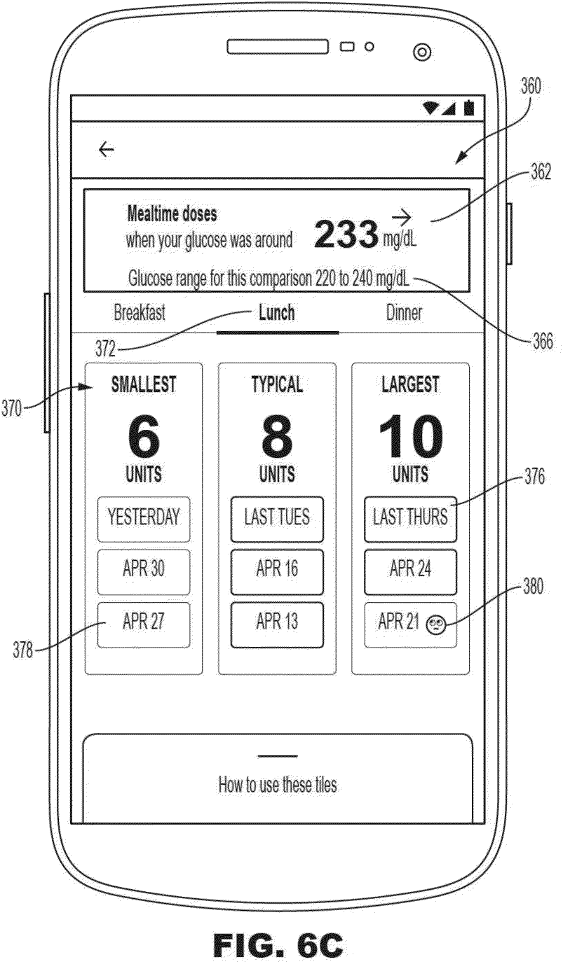

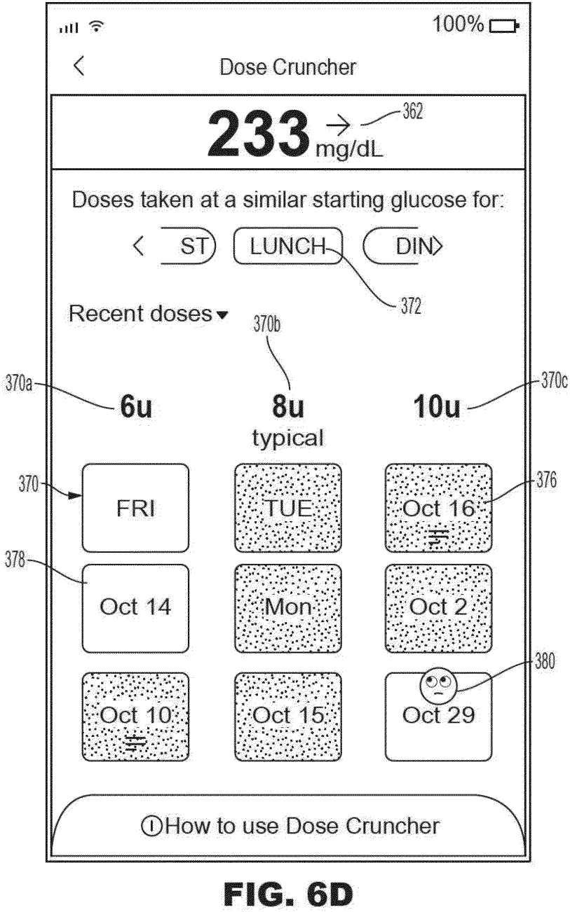

When a user is deciding how much medication, e.g., insulin, to take for the meal they intend to consume, as seen in FIG. 6A, the user can check their current analyte level 362 and can then can easily access the dosing pattern management application 300 from the sensor user interface application 330 by clicking a link 356, e.g., labeled “Check My Tiles.” Clicking the link 356 will open the home screen 360 (see FIGS. 6B, 6C, and 6D), which displays three dose groups, e.g., the smallest, typical, and largest dose amounts that the user has previously administered when their analyte level was around the current analyte level 362. As previously explained, the dose amounts that are displayed were doses that were administered when the user’s analyte level was around the current analyte level, i.e., within a defined analyte range 366 that includes the current analyte level 362. As seen in FIGS. 6B and 6C, the user can view the analyte range 366 of the doses displayed by clicking or selecting a link 364 labelled “around” to expand the screen to display the analyte range 366.

The past doses 370 can be displayed in groups, in which the smallest dose 370a, the typical dose 370b, and the largest dose 370c are all displayed for a particular meal 372 or time period of the day. The past doses display 370 can be displayed as tiles 374 that include the days, dates, or times when the particular dose was administered. The application can display at least one, alternatively at least two, alternatively at least three, alternatively at least 4 past dose tiles 374 per group per meal or time period.

Furthermore, for doses that resulted in positive results, e.g., the user reaching their goal range, the tile 374 can be highlighted by, e.g., coloring those entries a particular color (such as green) 376. Doses that did not result in bringing the user into their goal range can be colored a different color, e.g., gray or white 378. Additionally, if the dose resulted in hypoglycemia in the user within a period of time after administration (e.g., within 1 hour, alternatively within 2 hours, alternatively within 3 hours, alternatively within 4 hours), the gray tile will be additionally include a hypoglycemia marker 380, e.g., a hypoglycemia emoji. Users can toggle between the past doses displays for different meals 372 or time periods by selecting the different meal or time headings.

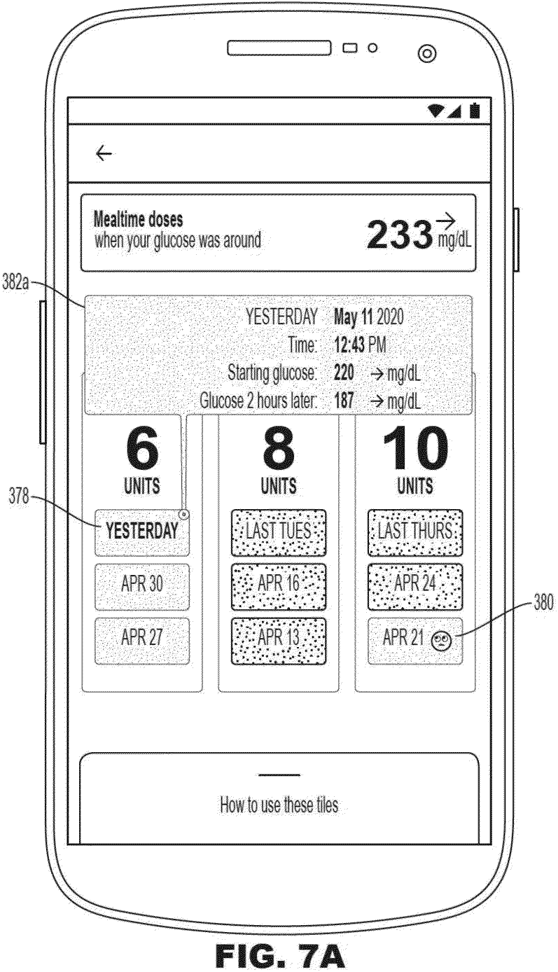

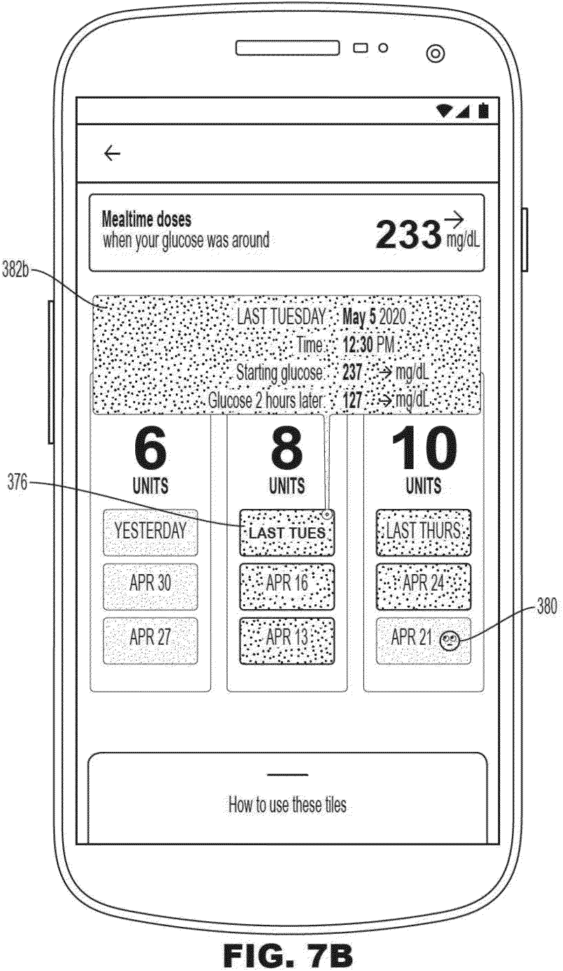

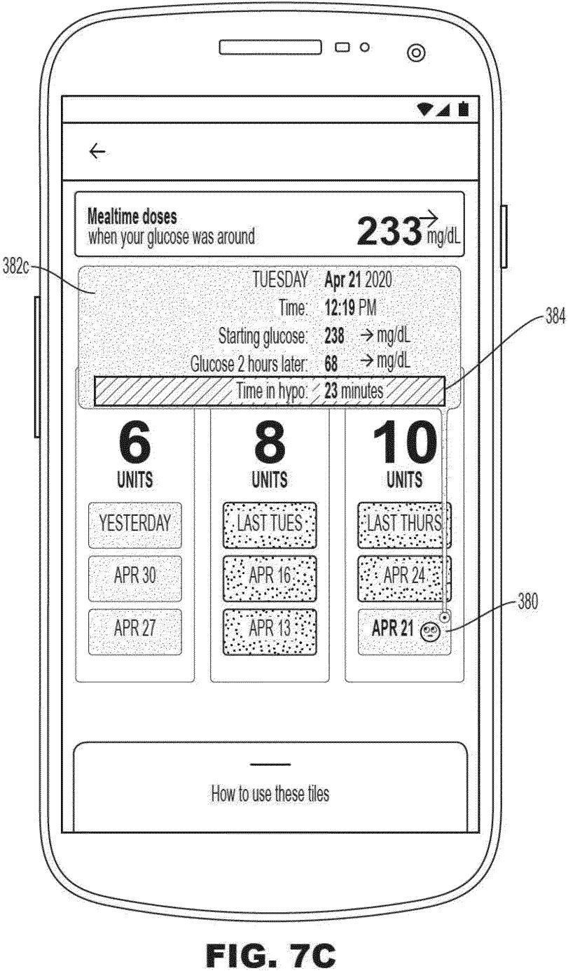

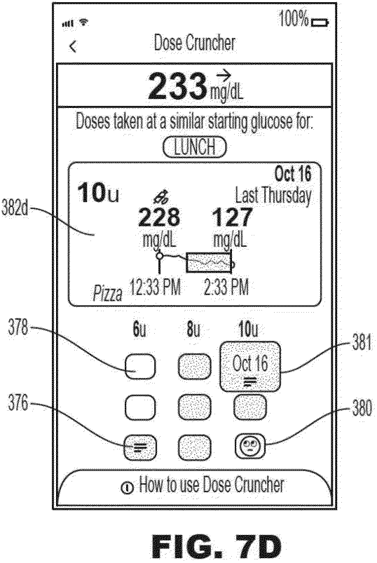

As seen in FIGS. 7A-7D, users may select or click on a tile 374 and a details screen 382a-c will appear that includes the date that the dose was administered, the time that the dose was administered, the starting glucose (i.e., analyte level when the dose was administered), the glucose 2 hours later, and the amount of time in hypoglycemia (where applicable). As seen in FIG. 7A, when a gray tile 378 is expanded, the details screen 382a shows that the glucose level 2 hours later was above the goal range (e.g., 187 mg/dL > 180 mg/dL). As seen in FIG. 7B, when a green tile 376 is selected, the details screen 382b shows that the glucose level 2 hours later was within the goal range (e.g., 127 mg/dL < 180 mg/dL). As seen in FIG. 7C, when a gray tile with a hypoglycemia marker 380 is selected, the details screen 382c shows that the glucose level 2 hours later was below 70 mg/dL (e.g., 68 mg/dL < 70 mg/dL). Moreover, details screen 382c displays the amount of time in hypoglycemia 384. The details screen 382a-c may also include a graph of the user’s glucose concentration over a predetermined time period or other details about the hypoglycemia event, such as when the hypoglycemia event occurred within the predetermined time period after administration and the lowest glucose level recorded during the predetermined time period. The predetermined time period to monitor for a hypoglycemia event may be about 4 hours, alternatively about 3 hours, alternatively about 2 hours, alternatively about 1 hour. Moreover, if an additional medication (e.g., insulin) injection was administered, the time period to monitor for a hypoglycemia event may be restarted based on the time of that the additional medication was administered because the application will not be able to discriminate as to which of the first or additional administrations resulted in hypoglycemia. As seen in FIG. 7D, which includes tiles of different colors as described in other embodiments (e.g., gray or white tiles 378, green tiles 376, and a gray tile with a hypoglycemia marker 380), a graphical display 382d of a glucose trace for the relevant time period may also be displayed, e.g., above the dosing tiles for the tile that was selected 381. The graphical display 382d may show the starting glucose at the time of administration and the ending glucose after a specific time period (e.g., about 2 hours), along with the dose amount.

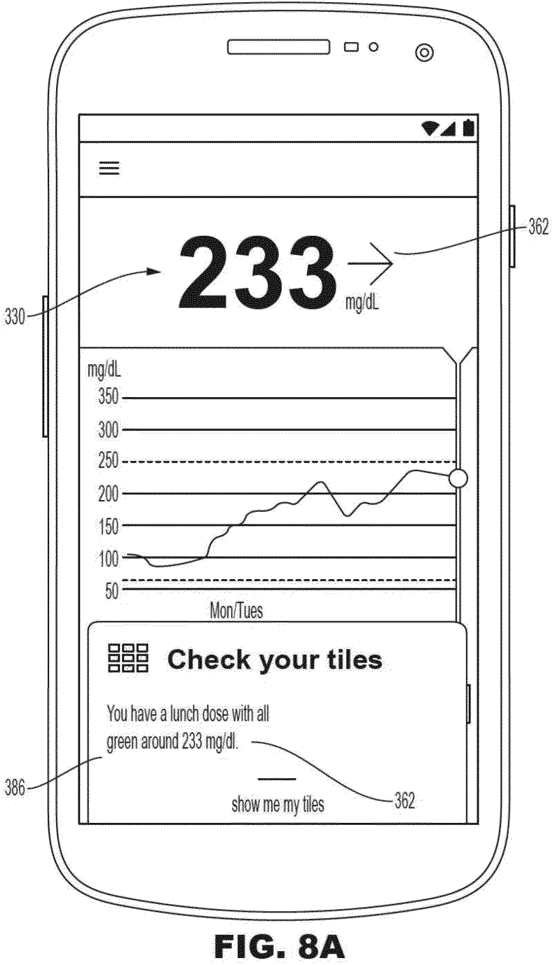

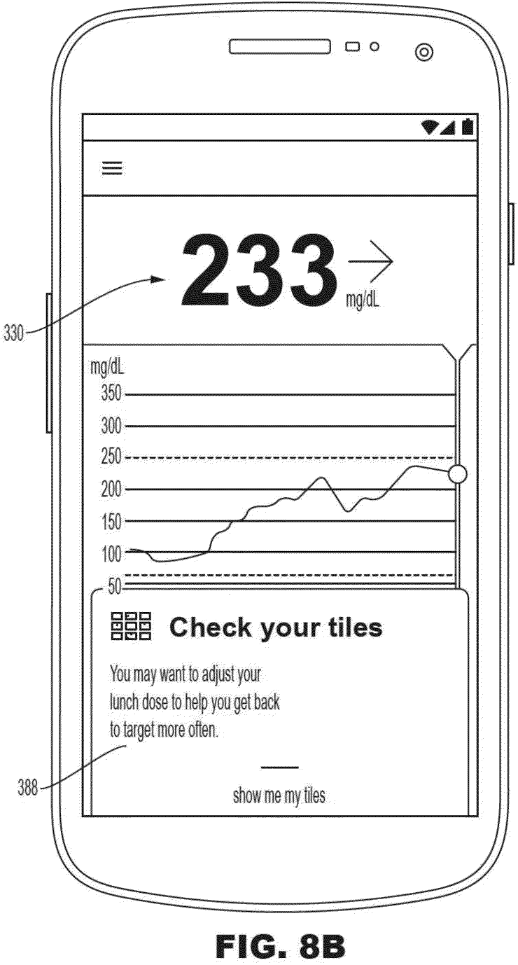

Windows or alerts may appear in the sensor user interface application or in a locked screen to prompt the user to check the tiles display in the dosing pattern management application 300. As seen in FIG. 8A, window 386 may appear to suggest that the user check their tiles because the user had previously administered a dose that resulted in the user reaching their goal range when the user’s analyte level was around the same as the current analyte level 362. Alternatively, as seen in FIG. 8B, the window 388 may contain a message suggesting that the user check their tiles and adjust a dose to help the user to get back on target.

### Star Ratings

In another embodiment, the dose pattern management application 300 can include a star rating system to help the user determine the best dose decisions. Doses that the user has taken in the past can get a start rating. Doses that got the user closest to their target or goal range after the meal have the most stars. For example, if a dose injected at breakfast got the user back to their target within 2 hours with no hypoglycemia, the dose would have 5 out our 5 stars. If, however, the dose resulted in a glucose level of 200 mg/dL 2 hours after administration, then the dose may be rated with only 4 out of 5 stars because the user came close but did not reach their goal range of under 180 mg/dL. Moreover, if the user experiences hypoglycemia within 4 hours of administration, stars will be deducted from the rating.

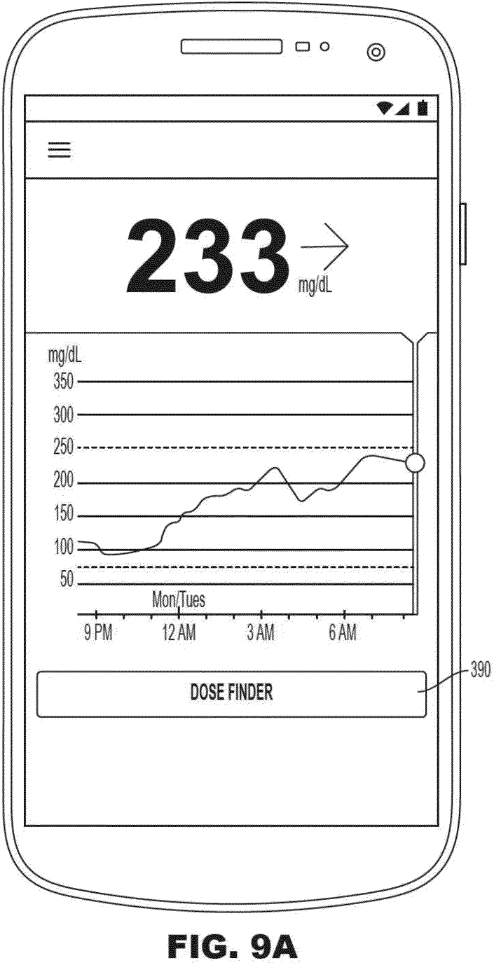

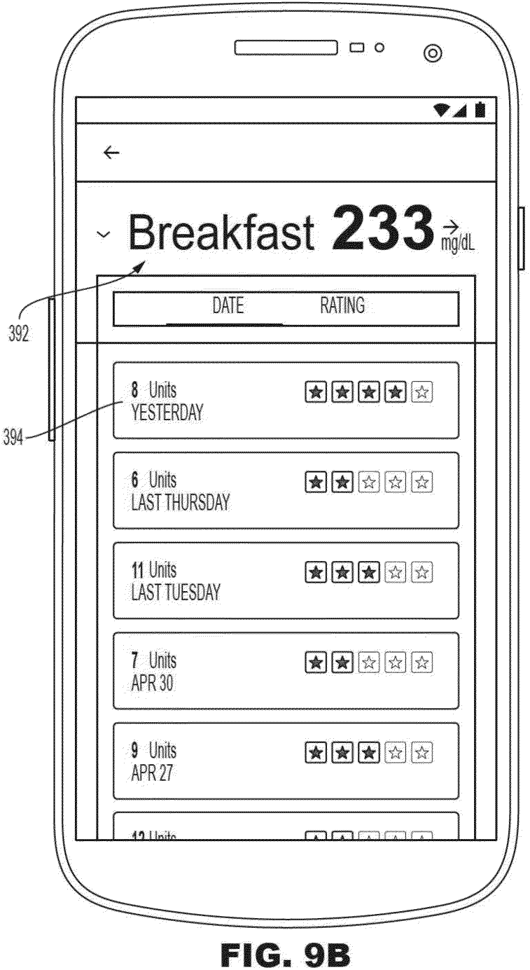

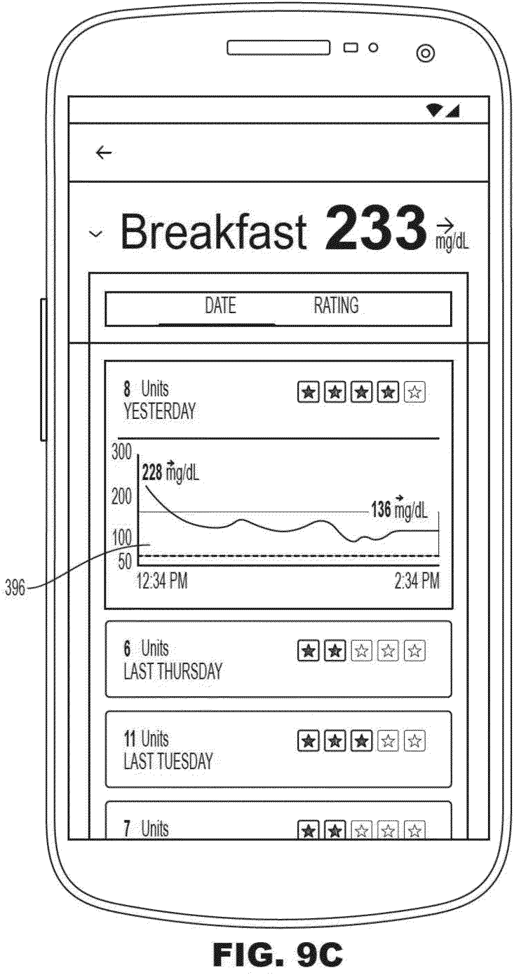

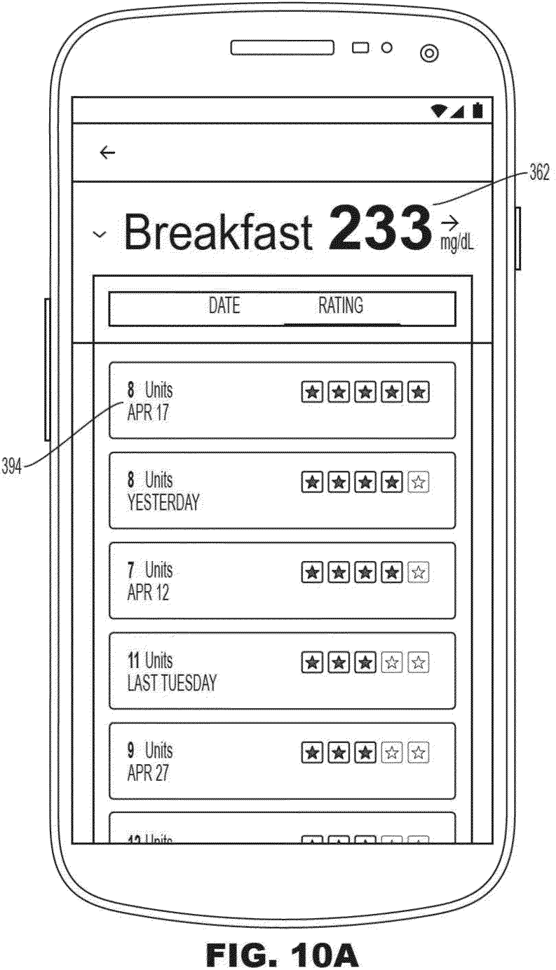

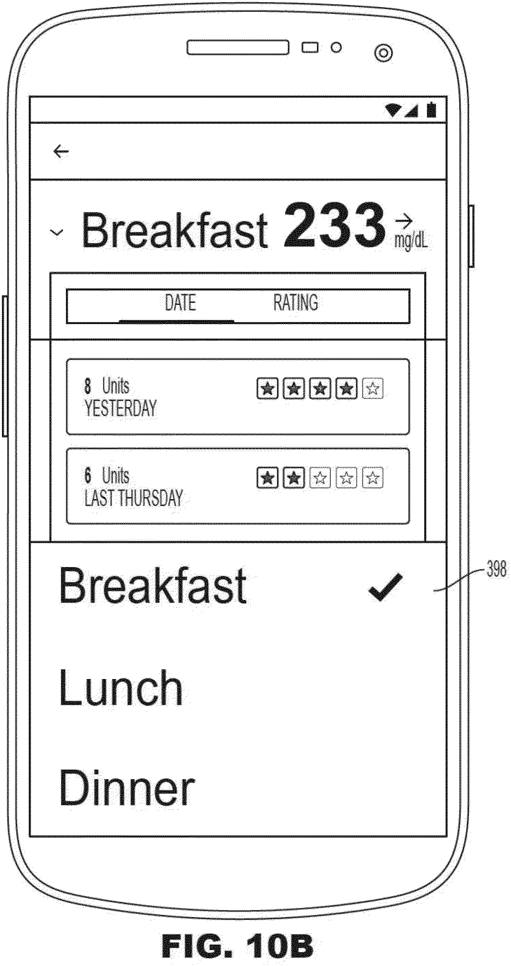

As seen in FIGS. 9A and 9B, clicking on the link 390 opens a home screen window 392 that displays doses organized either by date or rating. Unlike some other embodiments, similar dose amounts may not be grouped together in this view. The dose entries 394 include the amount of the dose, the day or time the dose was administered, and the rating. When the user clicks on the dose entry 394, the entry can additionally expand to show a graph 396 of the glucose concentration from the time of administration to a period of time after administration (e.g., about 2 hours after administration). The dose entries 394 can be sorted by date (see FIGS. 9B and 9C) or by rating (see FIG. 10A), e.g., highest number of stars first. As seen in FIG. 10B, the user can also select different meals 398 to display different doses associated with the selected meal.

### Tags

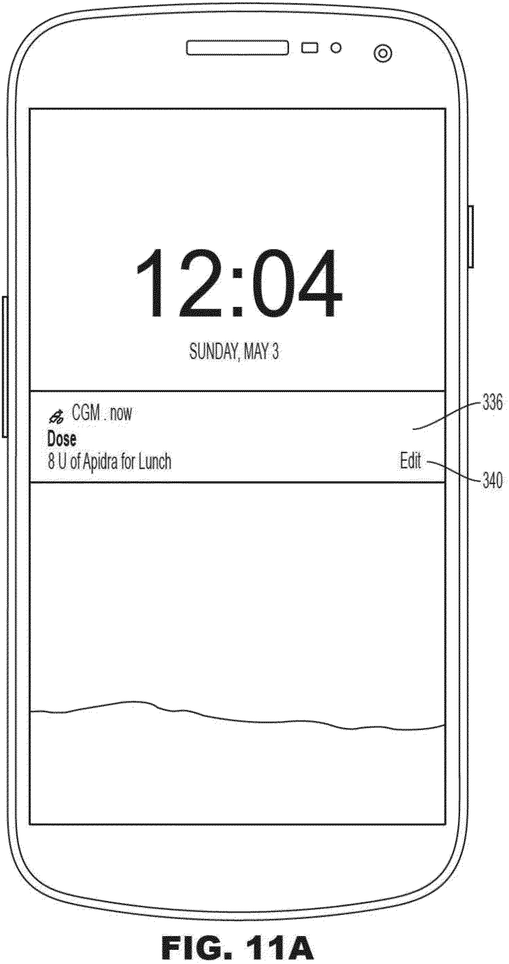

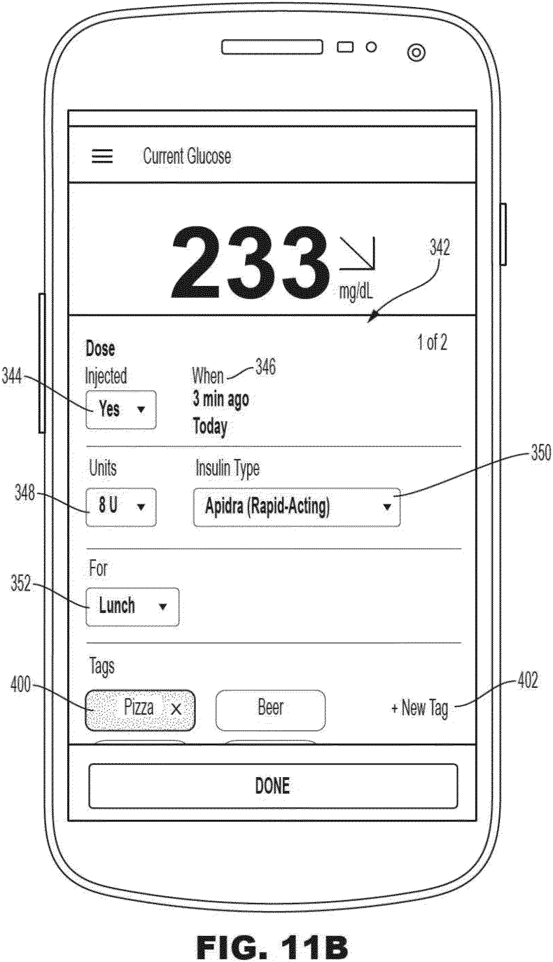

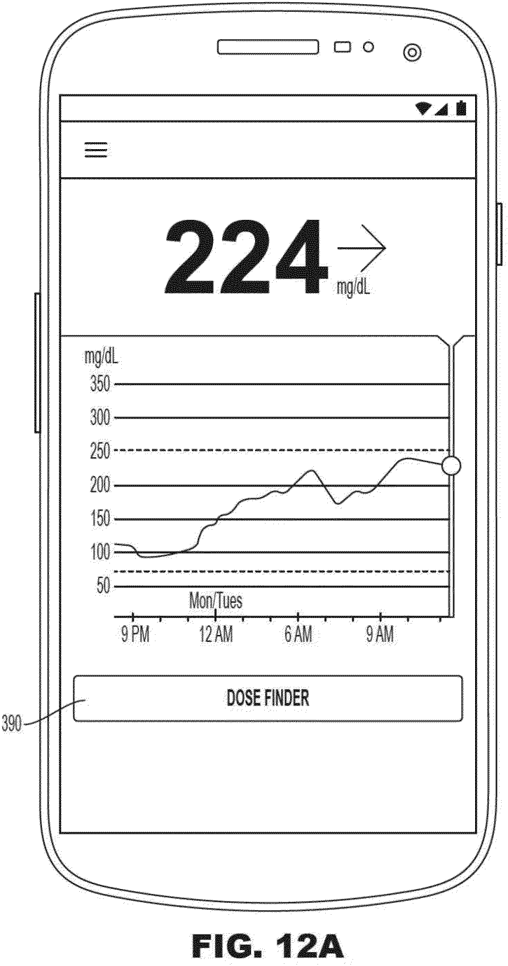

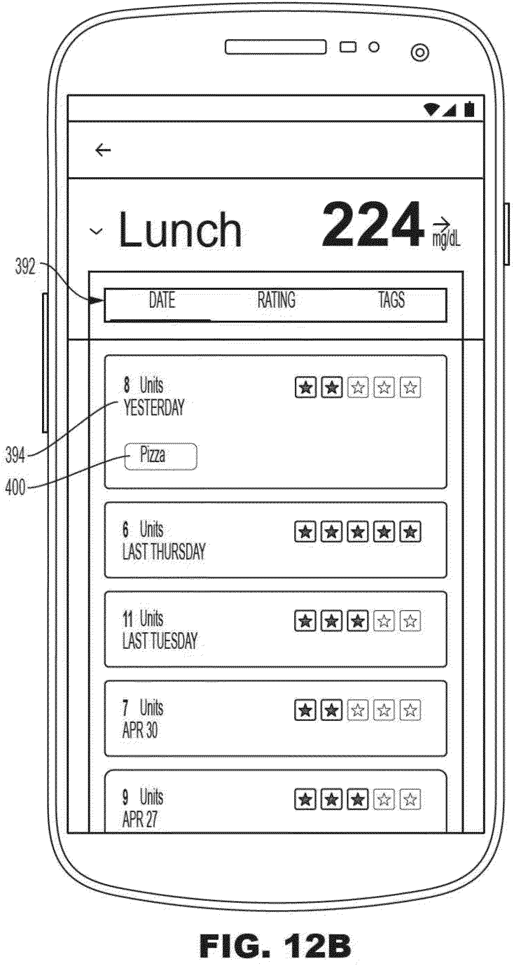

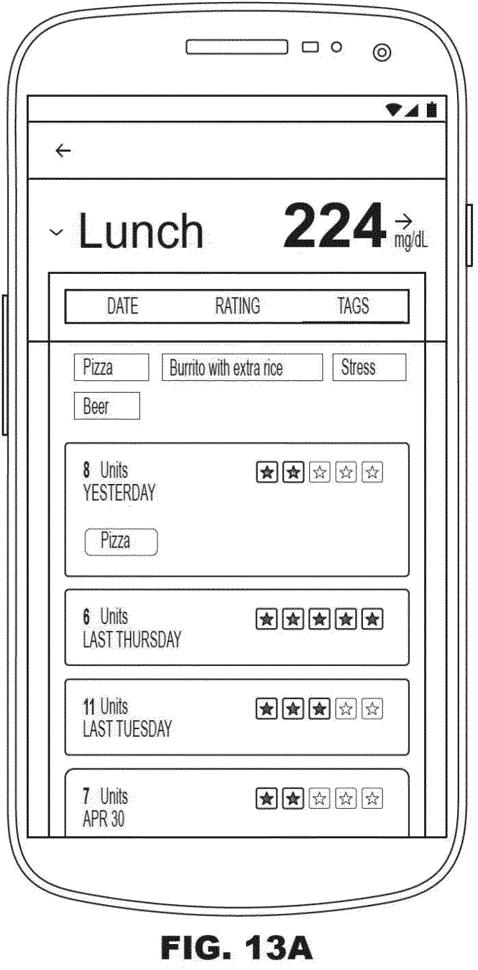

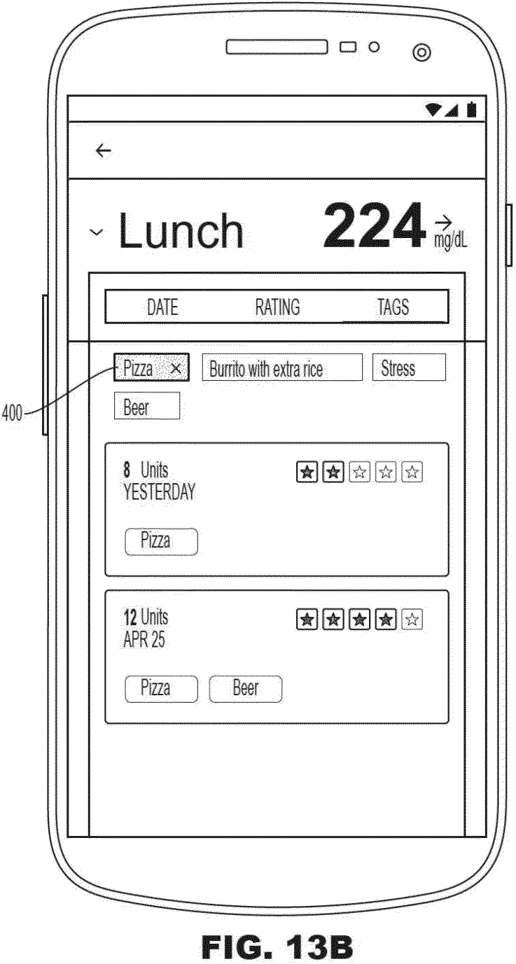

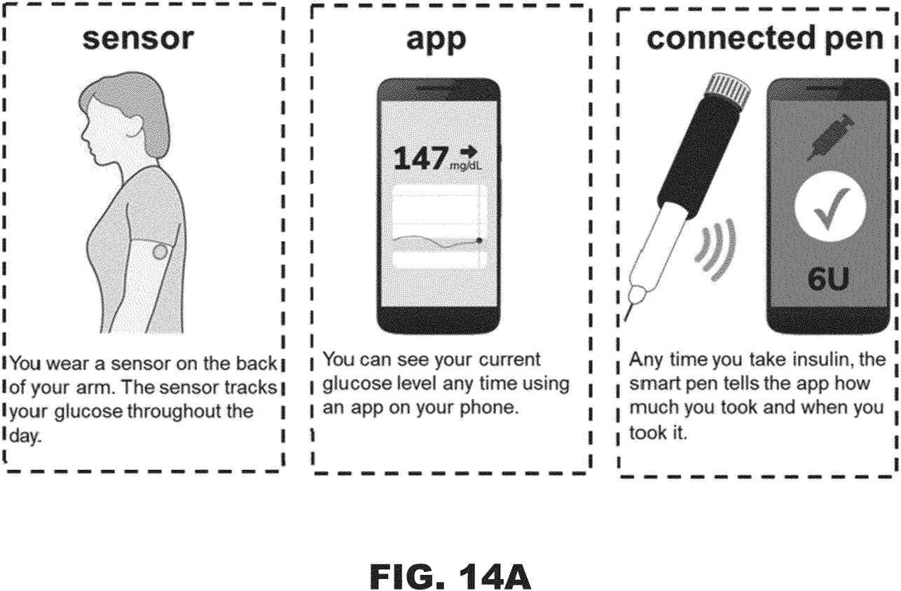

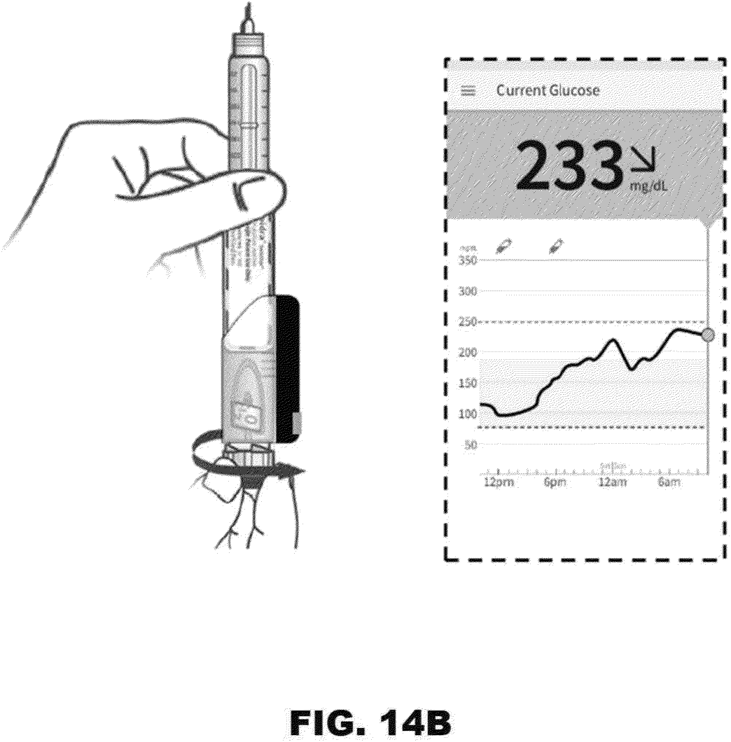

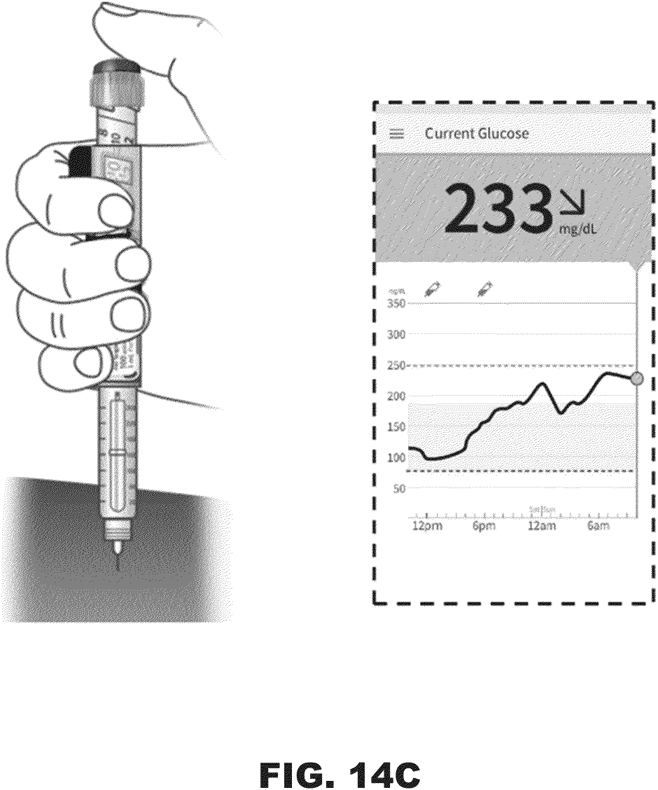

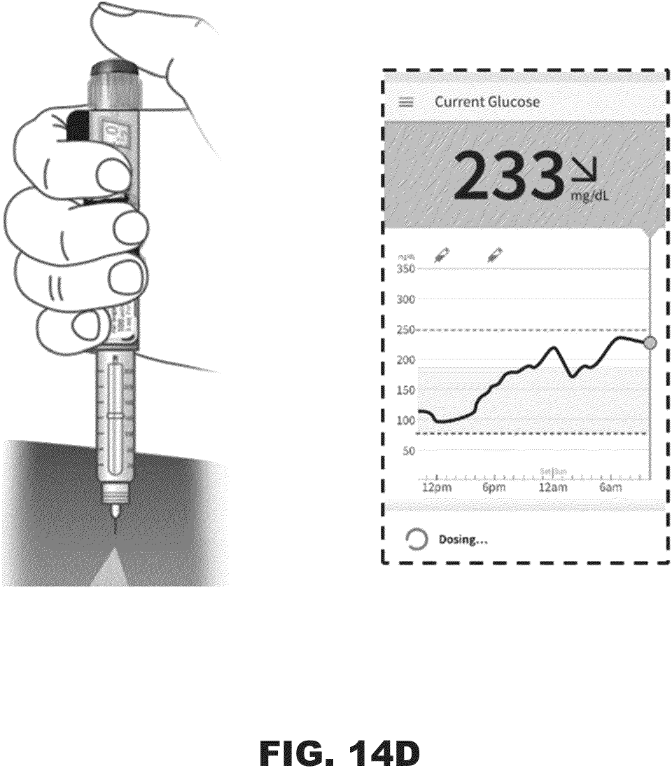

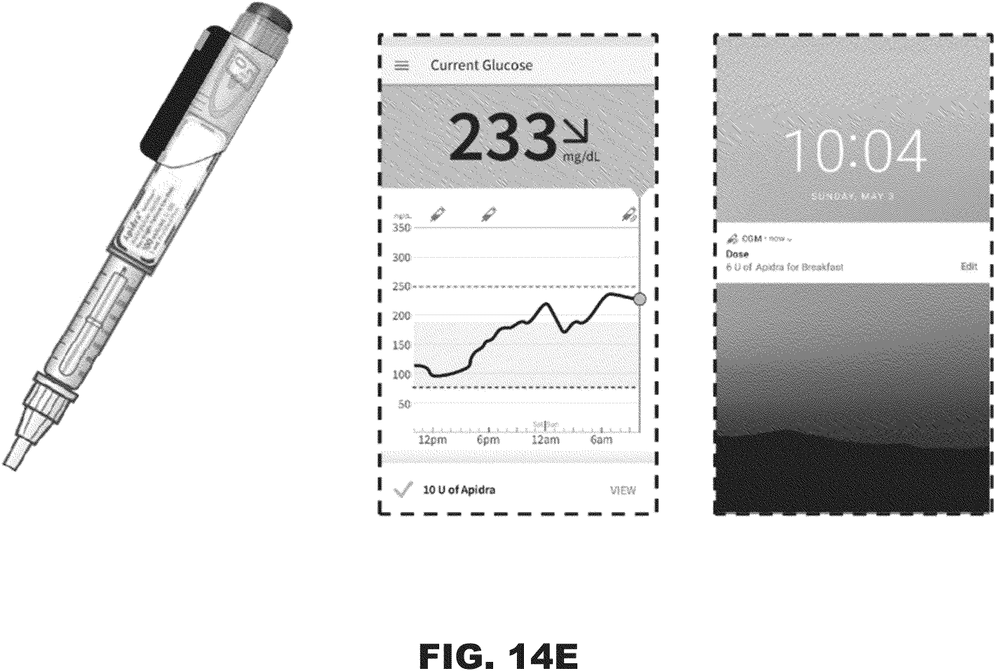

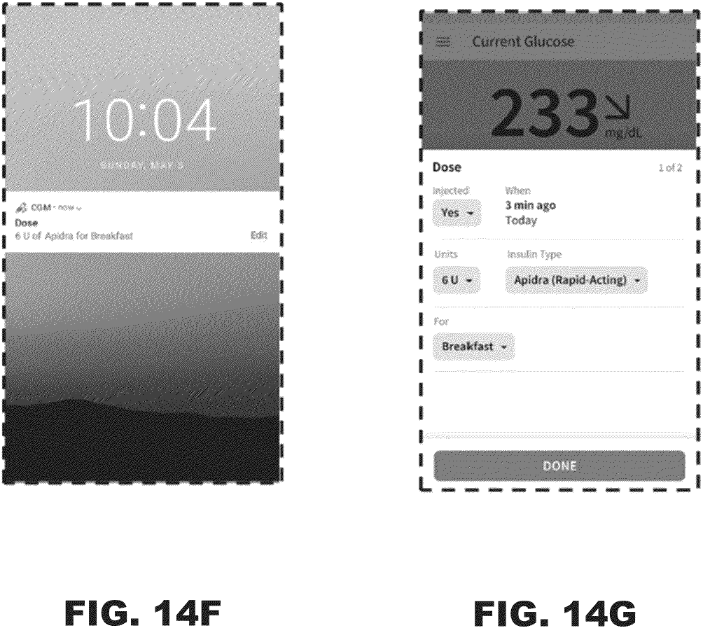

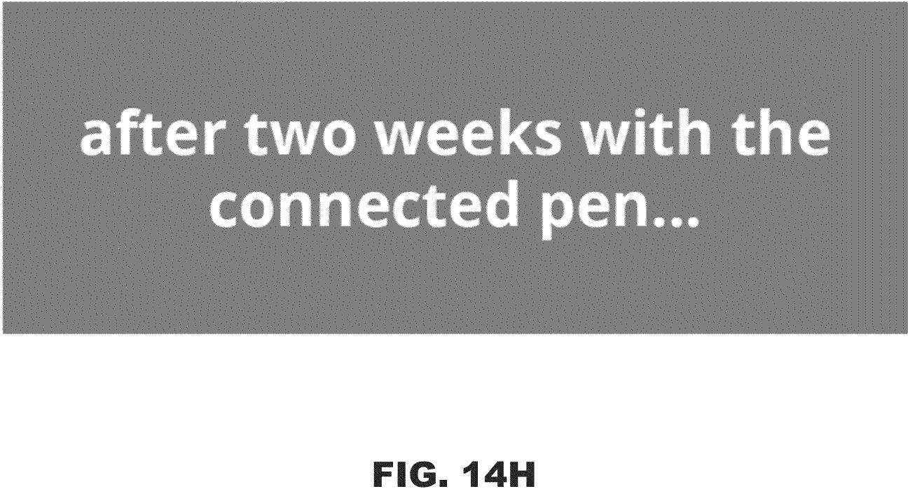

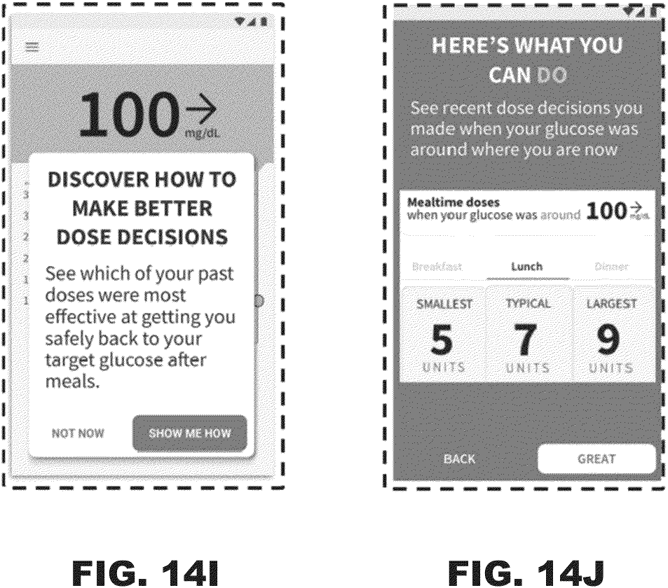

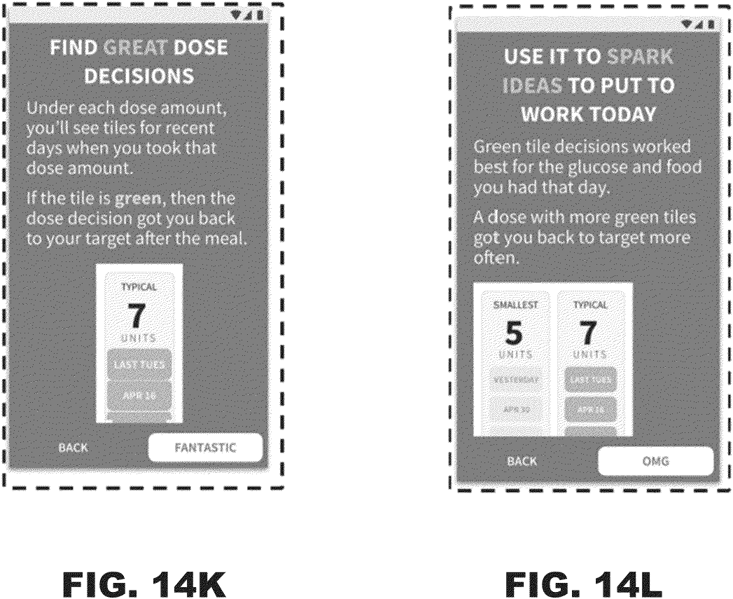

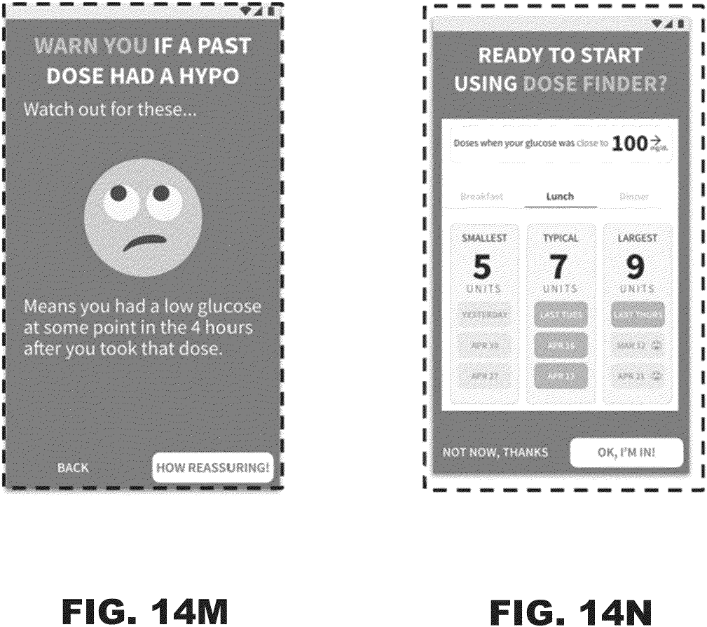

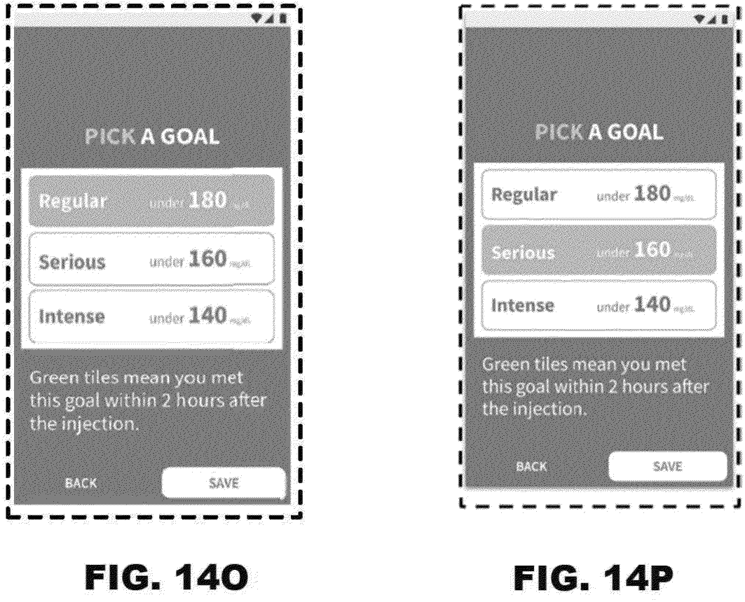

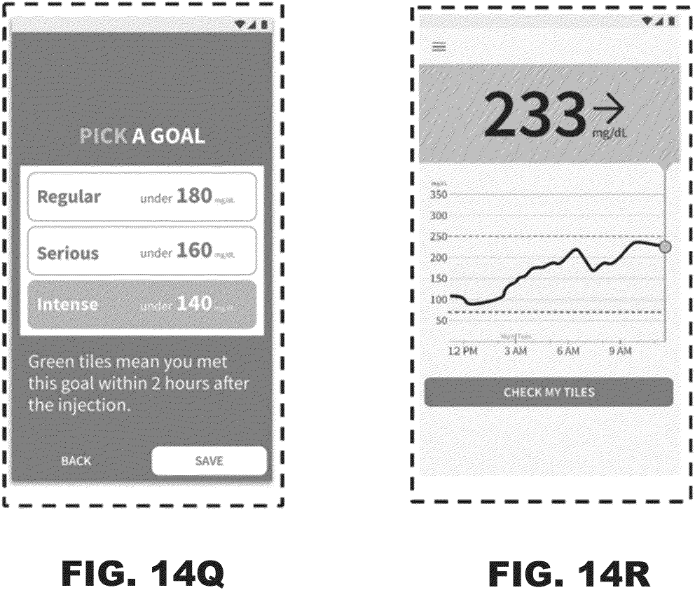

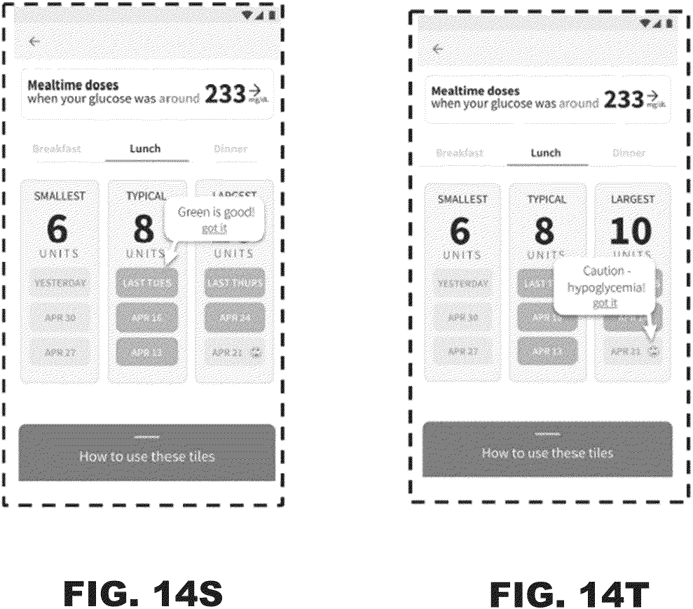

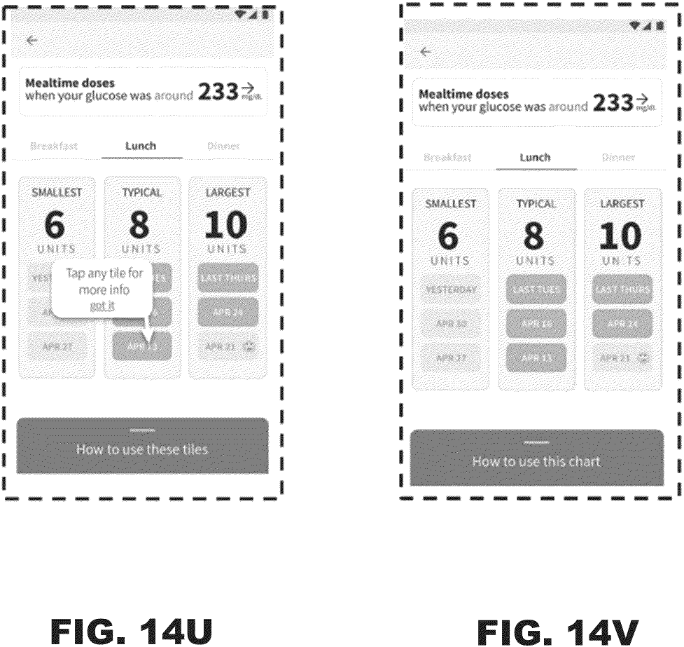

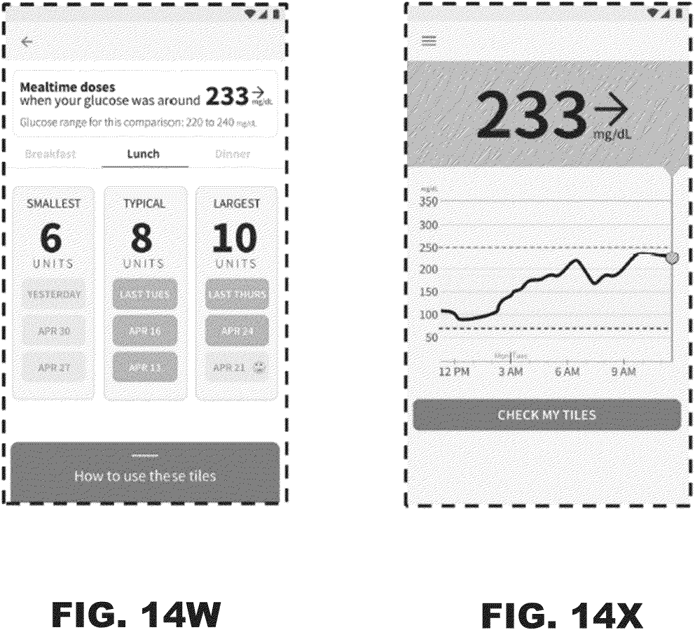

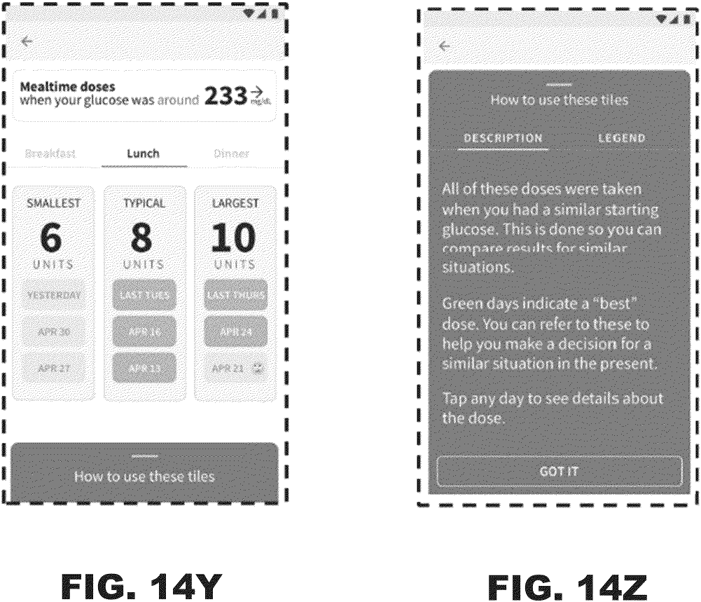

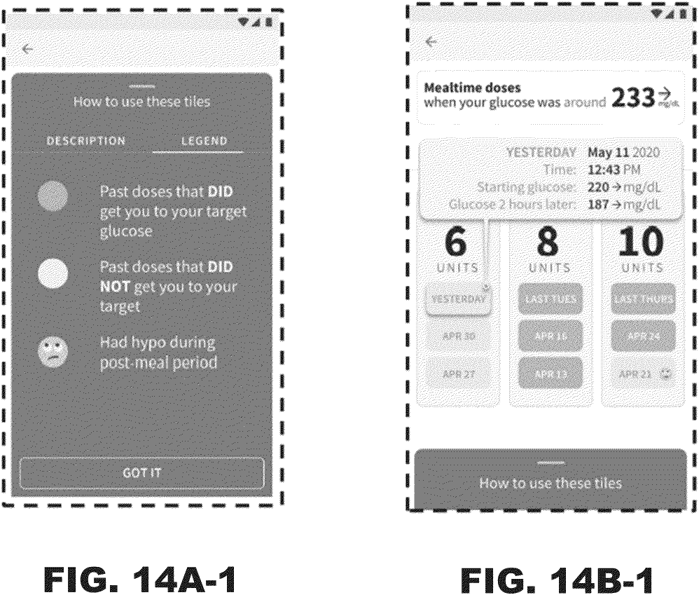

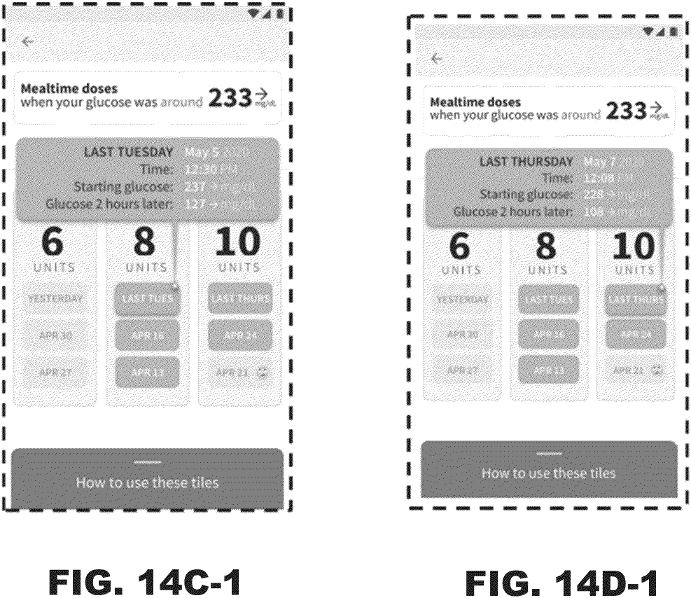

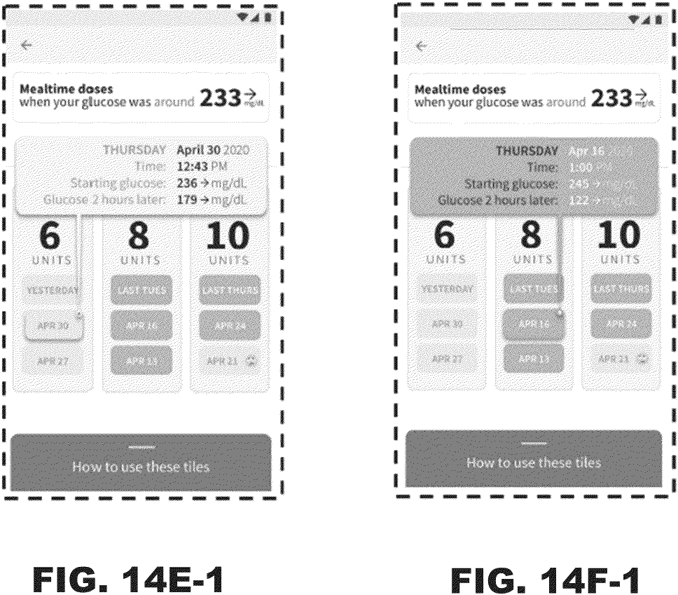

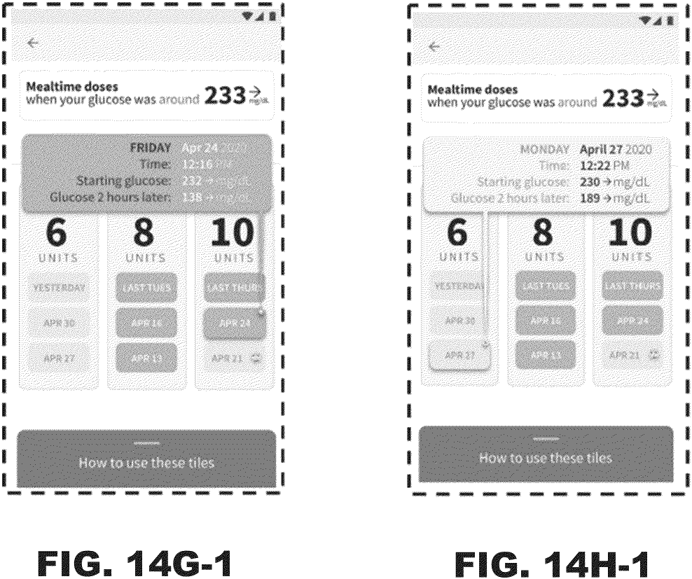

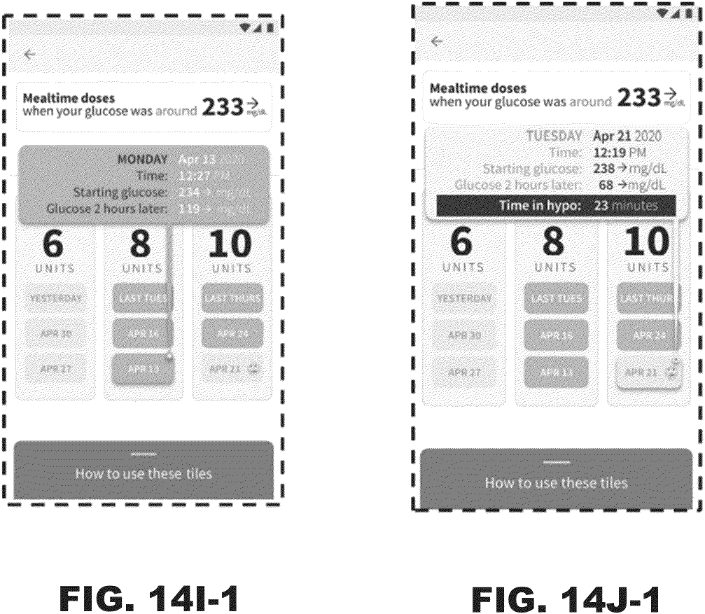

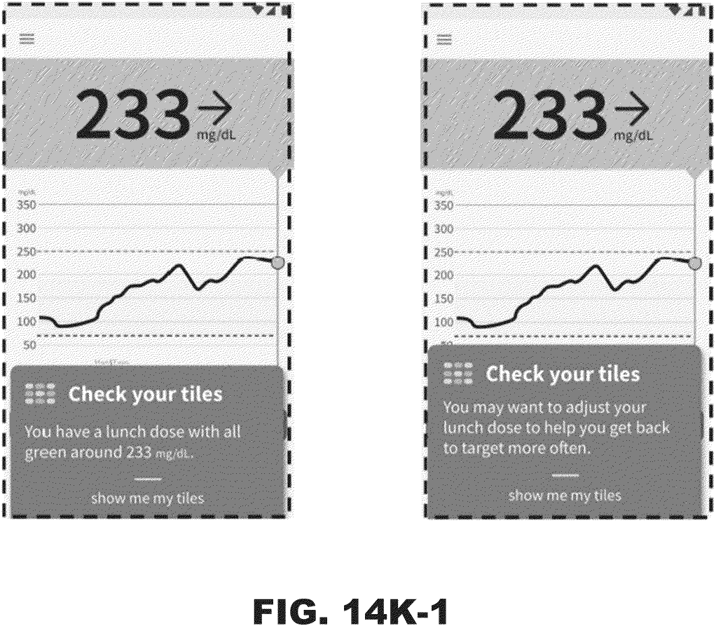

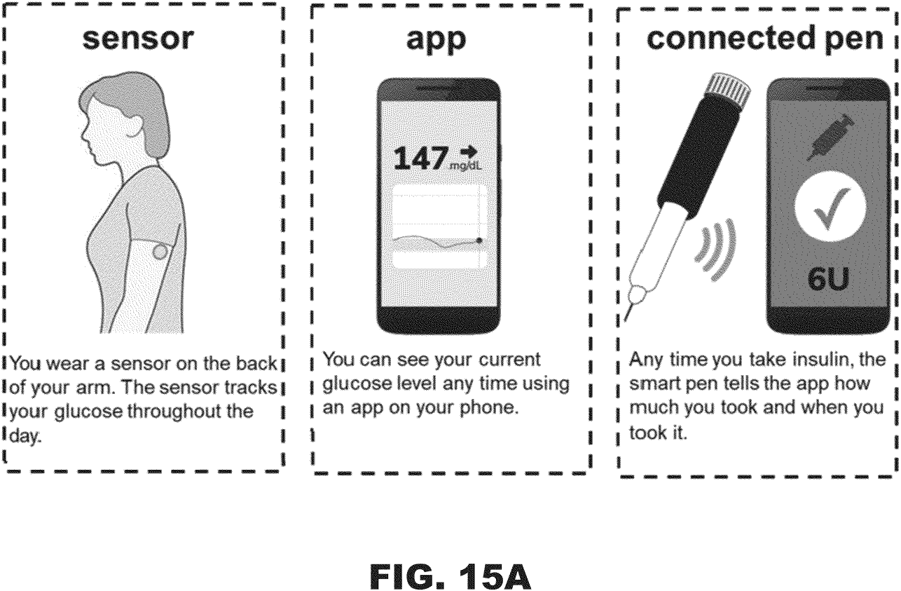

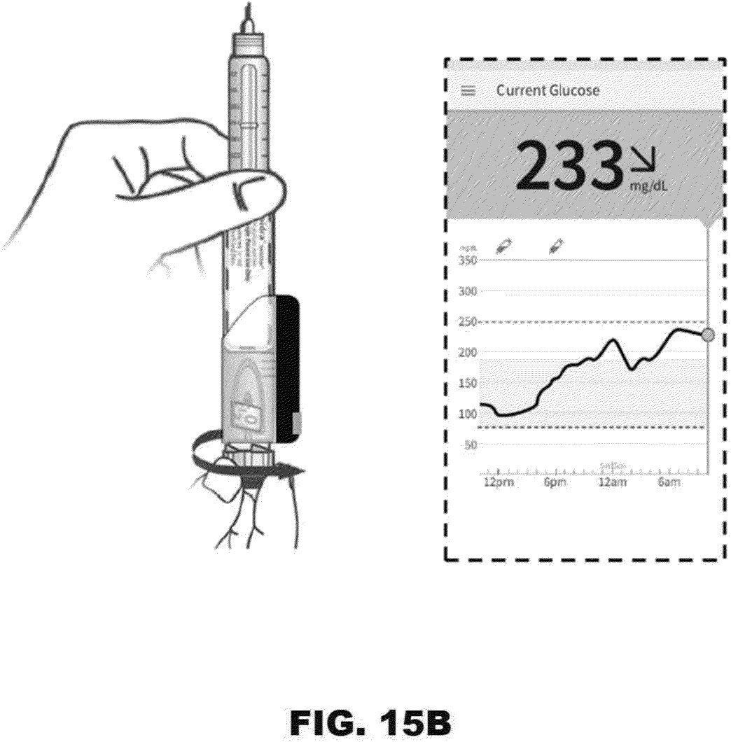

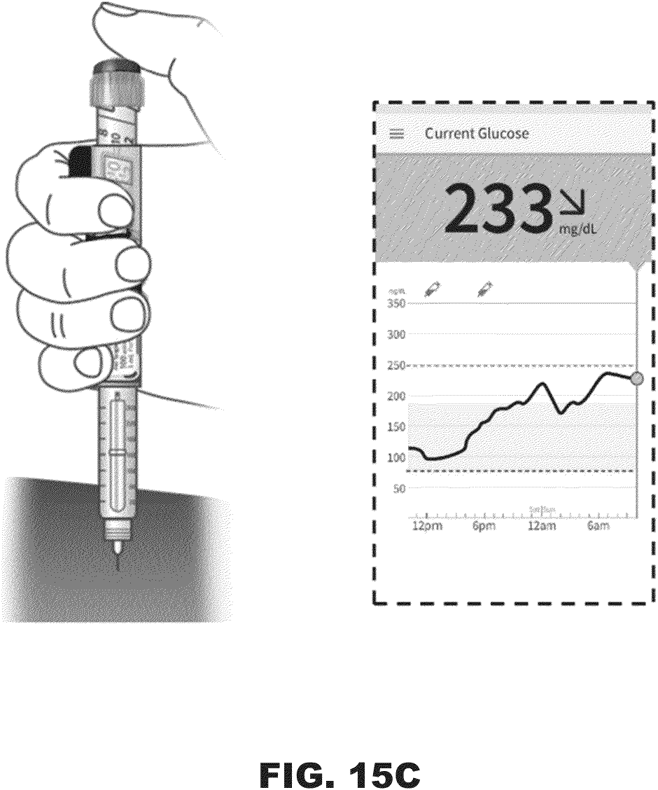

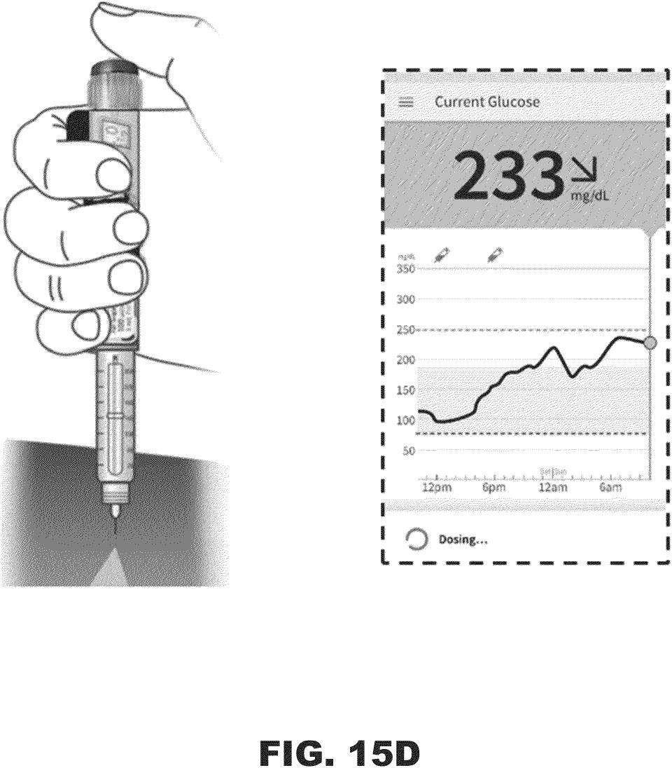

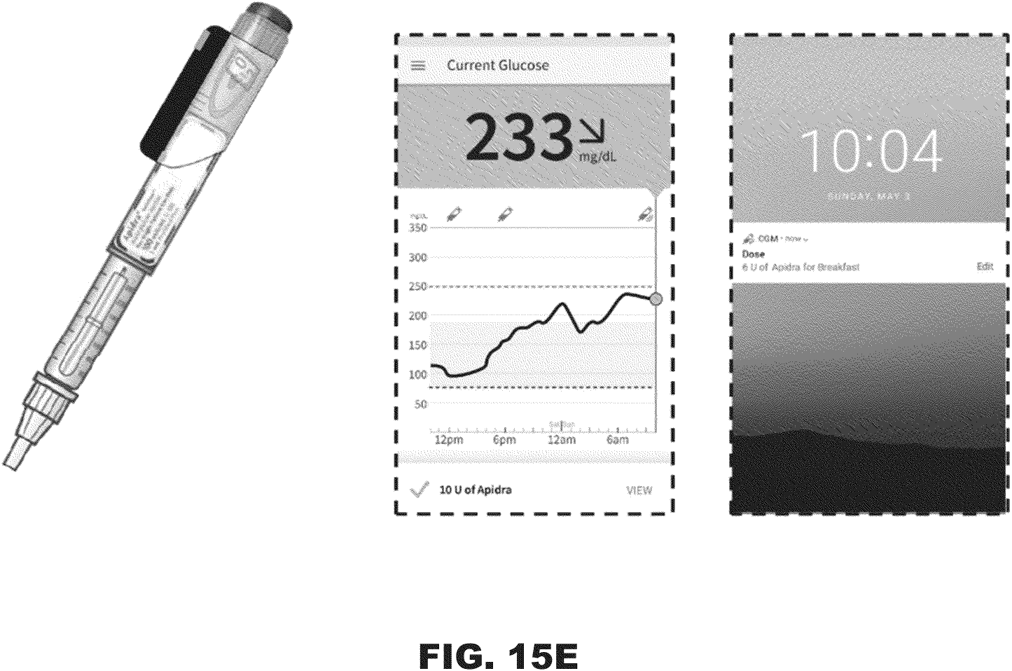

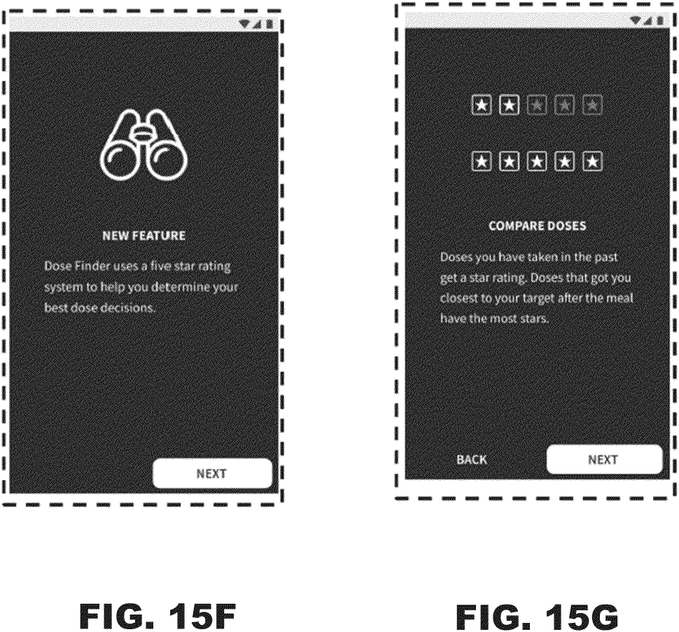

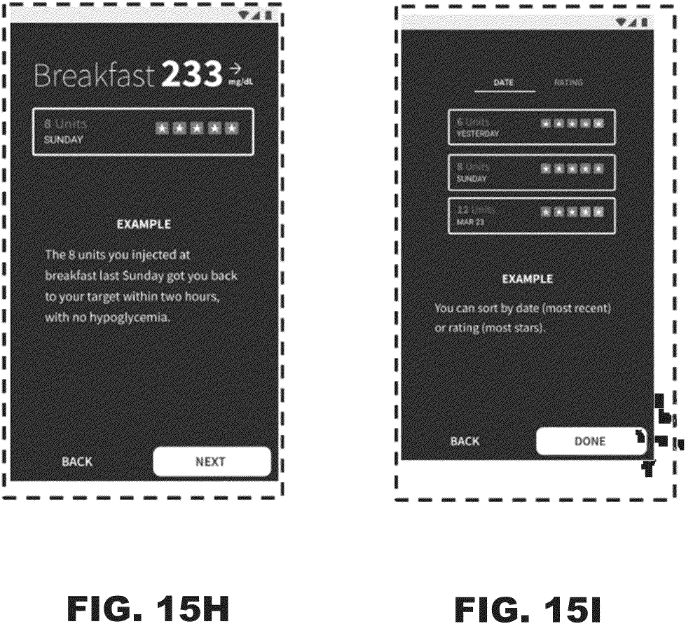

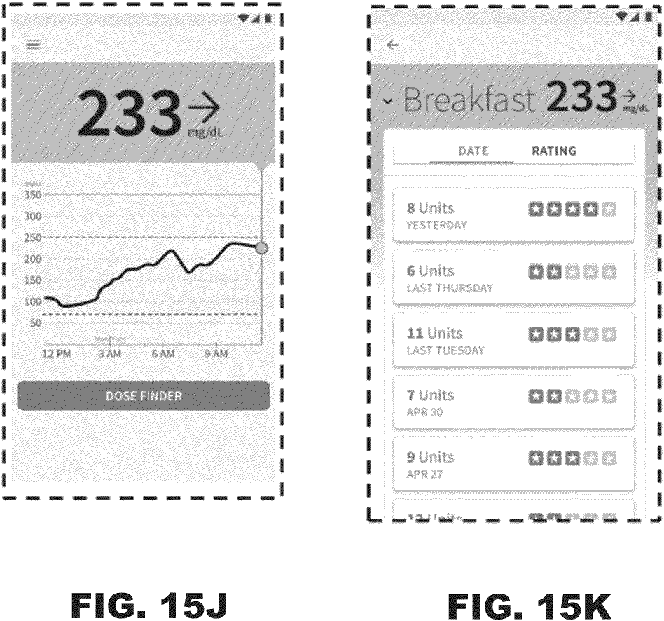

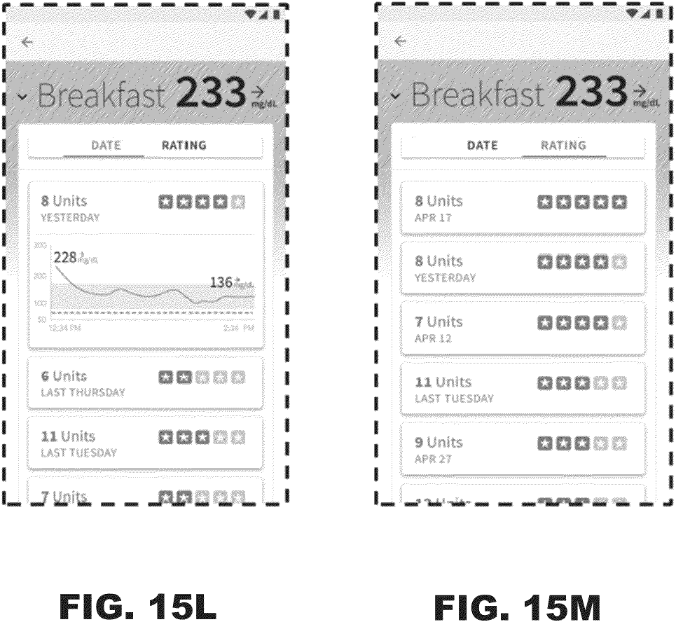

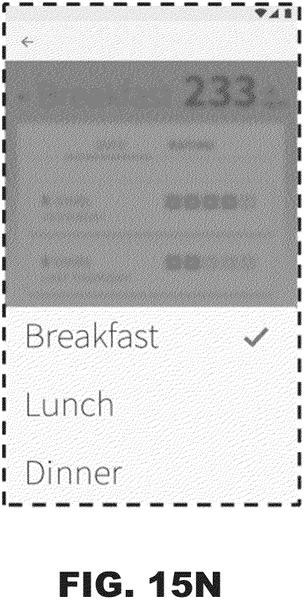

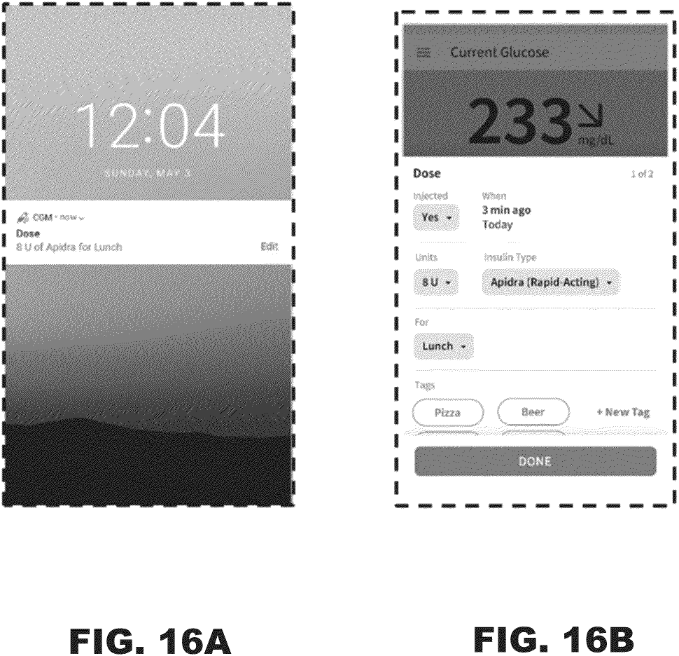

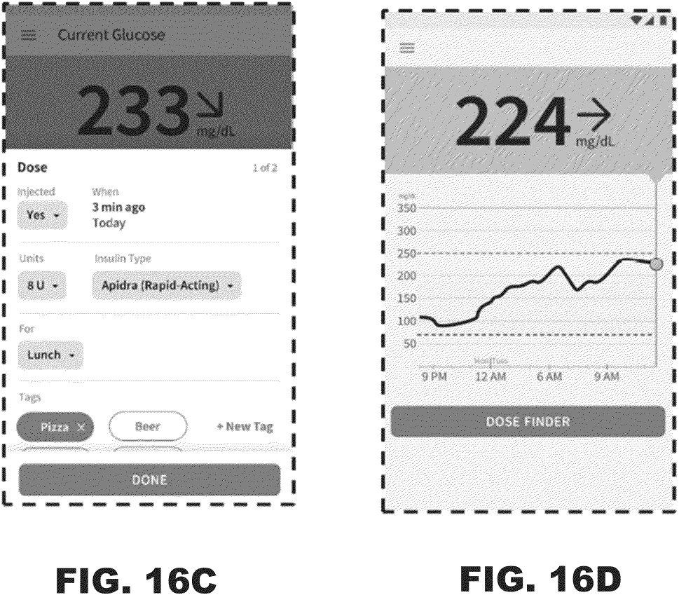

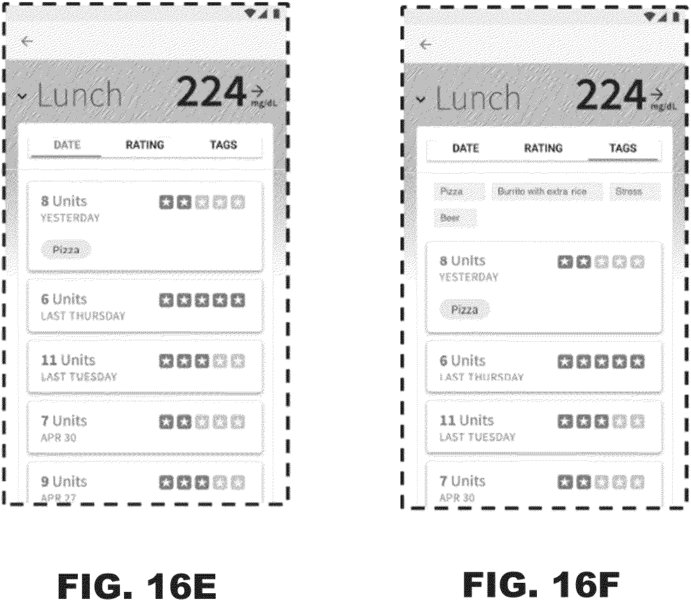

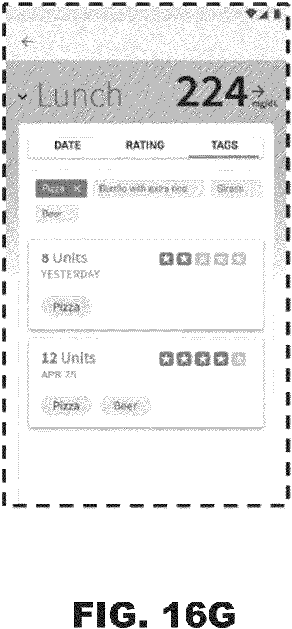

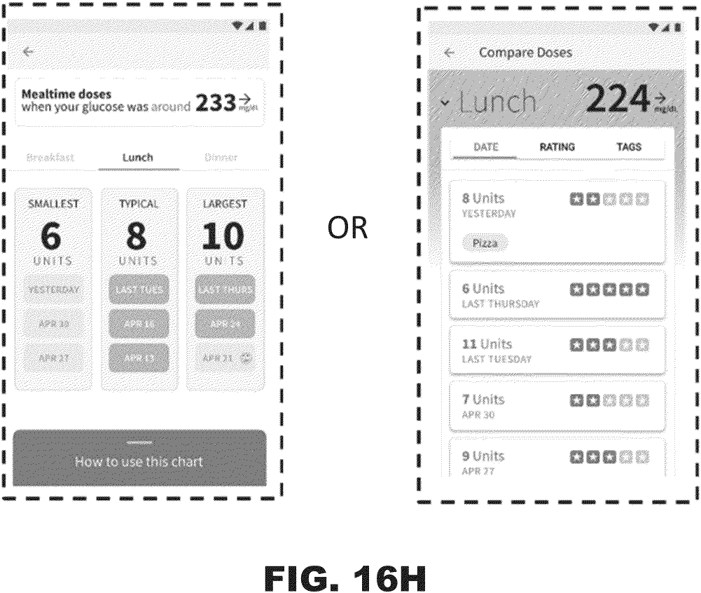

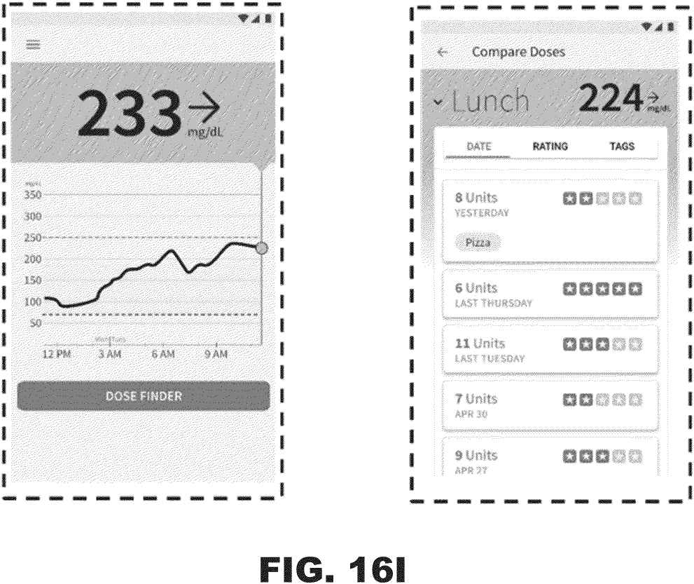

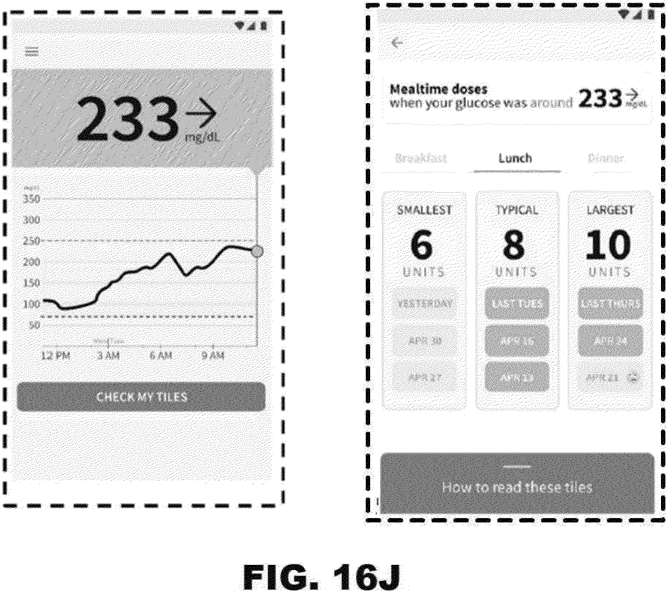

In another embodiment, the dose pattern management application 300 can include tags 400 that the user can associate with a dose entry, which could help jog the user’s memory as to the circumstances surrounding a particular dose. As seen in FIGS. 11A and 11B, the user can click the link 340 (e.g., “edit”) on the dose alert 336 or simply tap the notification to open the dose details screen 342. In addition to the various fields described previously that the user can edit, including method of administration (Injected - Yes/No) 344, time of administration 346, amount of medication administered 348, type of medication 350, and the corresponding meal with which the medication was administered 352, the user can also add a tag 400 to the dose entry. The tag 400 may be a preexisting tag or the user can add a new tag 402. The tags 400 may be associated with a particular food, or may not be related to food (e.g., stress or exercise). As seen in FIGS. 12A and 12B, clicking on the link 390 opens a home screen window displaying a dose details screen 392 that displays doses organized either by date, rating, or tags 400. Unlike some other embodiments, similar dose amounts may not be grouped together in this view. The dose entries 394 include the amount of the dose, the time the dose was administered, the rating, and the tags 400. The dose entries 394 can be sorted by date (see FIG. 12B), by rating, or by tags (FIGS. 13A and 13B). The user can also select different meals to display different doses associated with the selected meal.

### Suggested Doses and Ranges

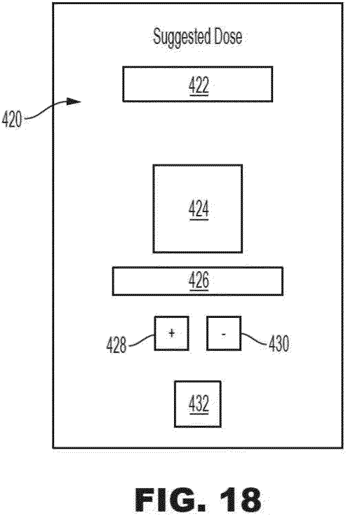

In another embodiment, the dose pattern management application 300 may display a suggested dose right now. As seen in FIG. 18, a suggested dose interface 420 may include the name of the meal 422 for the suggested dose, the suggested dose 424, an indication that the dose corresponds to a typical meal dose 426, links “+” 428, “-” 430 that the user can select to increase or decrease the suggested dose, and a home button 432. The name of the meal 422 for the suggested dose may list the meal name and may also list that it is for the current meal, e.g., “breakfast, “lunch,” “dinner,” “breakfast right now,” “lunch right now,” or “dinner right now.” The dose amount 424 may be prominently displayed as an amount of suggested units to administer, e.g., “10u.” Below the suggested dose amount 424, there may be an indication 426 that this dose is for a “typical” breakfast, lunch, or dinner. The suggested dose amount 424 may be determined as described in, e.g., U.S. Application Serial No. 16/944,736, published as U.S. Pat. Publication No. 2021/0050085, which is hereby expressly incorporated by reference in its entirety.

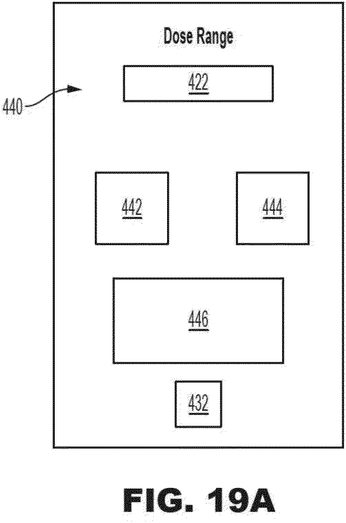

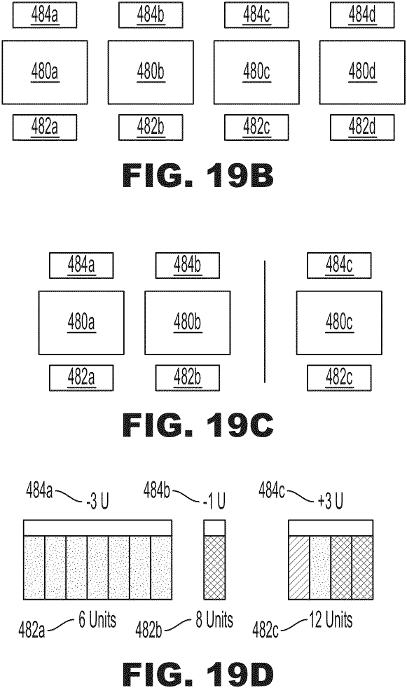

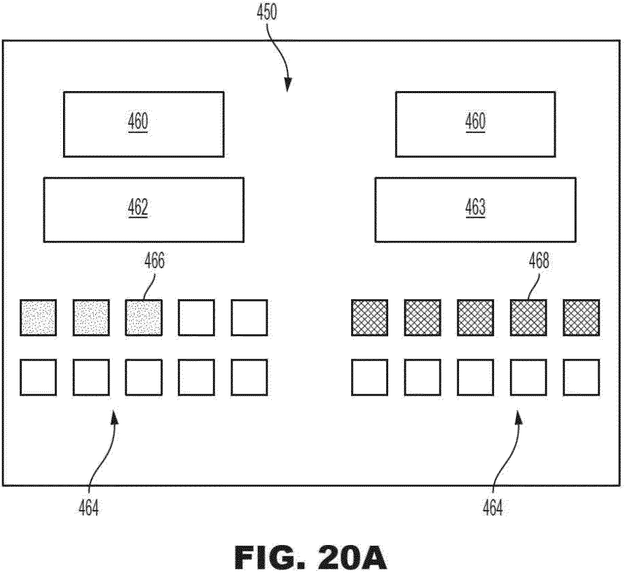

The dose pattern management application 300 may display a dose range for a particular meal. As seen in FIG. 19A, a dose range interface 440 may include the name of the meal 422, a lower dose amount 442, an upper dose amount 444, an explanation 446 of the safe range displayed, and a home button 432. The name of the meal 422 for the suggested dose may list the meal name and may also list that it is for the current meal, e.g., “breakfast, “lunch,” “dinner,” “breakfast right now,” “lunch right now,” or “dinner right now.” The lower suggested dose amount 442 and higher suggested dose amounts 444 may each be displayed as a number of units. The safe range interface 440 may also indicate that the lower suggested dose amount 442 be taken with a meal with fewer carbohydrates and the upper suggested dose amount 444 be taken with a meal with more carbohydrates. The explanation 446 may indicate that the doses displayed are the lowest and highest suggested doses and that the user can decide how much to take within this range based on food and exercise.

The dose pattern management application 300 may also generate a graphical display that visually indicates the user’s response to various different doses within the safe range. As seen in FIGS. 19B-19D, different graphical display 480a-d reflecting whether the dose was good or bad may be grouped together by dose amounts 482a-d (e.g., X units). The difference in units as compared to the suggested dose 484a-d may also be displayed (e.g., -3 U, -2 U, -1 U, +1 U, +2 U, +3 U). As seen in FIG. 19D, the graphical displays 480a-d may include an entry for each time a dose of that amount was given (e.g., four boxes if four injections of that amount were administered). For each dose administered, the graphical display may be color coded to indicate if the dose was determined to be a good or bad dose, as described elsewhere in the application. The current day of the month may be highlighted with a dot or different color.

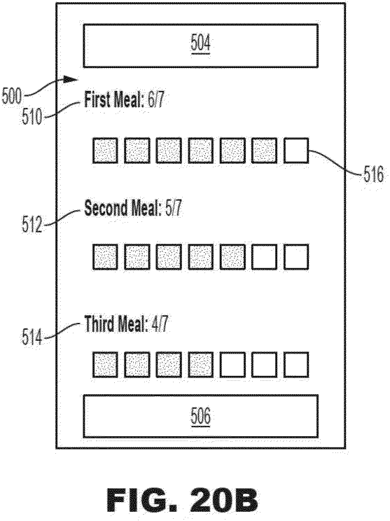

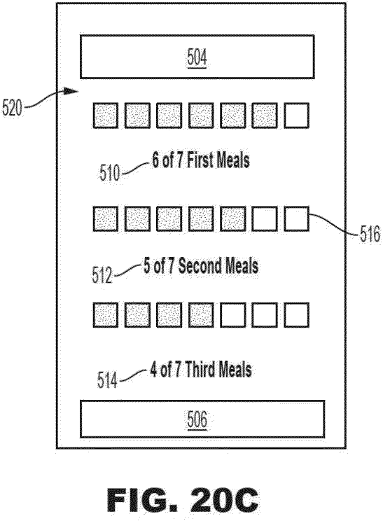

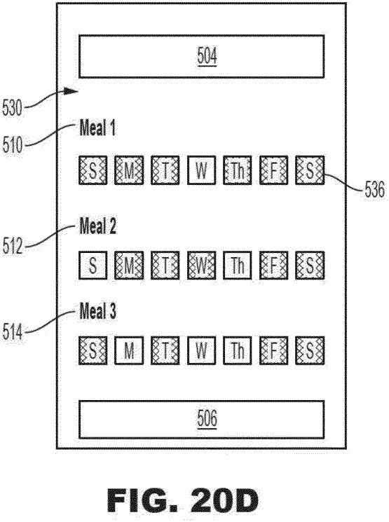

The dose pattern management application 300 may also generate a display or report that indicates in text and graphs how often the user’s injections have met or almost met the user’s post-meal target goal. As seen in FIG. 20A, the display or report 450 may include a metric or an indication of how many injections met the user’s post-meal glucose target such as X injections in Y total, e.g., “3 in 10” or “5 in 10.” The display may also indicate if the metric is for injections that met or almost met the post-meal target 432 or for injections that met the post-meal glucose target 463. The display or report 450 may also include a graphical or pictoral display of how many injections met or almost met the goal. For instance, a display of a grouping of icons 464 such as squares, circles, apples, bowls, etc., can be displayed. A proportion related to the metric 460 reported may be shaded. For example, if 3 in 10 injections met or almost met the goal, then a group of 10 icons (e.g., apples) may be displayed and 3 of the 10 icons may be shaded. The icons indicative of injections having met the post-meal goal may be the same or a different color than the icons indicative of injections having met or almost met the goal.

In another embodiment, as seen in FIGS. 20B-20D, the display or report 500, 520, 530 may include a metric or an indication of how times the user’s post-meal glucose target was reached such as X meals in Y total, e.g., “3 in 7” or “5 in 7.” The metric or indication 516 may be displayed for at least one meal (breakfast 510, lunch 512, and/or dinner 514), or alternatively, all three meals for a given time period. The time period may be a week, a month, a plurality of months (e.g., 3 months), or a year. For example, if the user reached their post-meal glucose target for 3 of 7 days after a meal (e.g., breakfast), then a group of 7 icons 516 (e.g., celebratory emojis) may be displayed and 3 of the 7 icons may be highlighted (e.g., shaded or bolded) while the other 4 icons may not be highlighted. The icons indicative of meals having met the post-meal goal may be the same or a different color than the icons indicative of injections having met or almost met the goal. The metric may be reported as “X/Y” as in FIG. 20B or as “X of Y” as in FIG. 20C. Alternatively, as seen in FIG. 20D, the group of 7 icons 536 may correspond to the days of the week and the icon corresponding to the day may be shaded or otherwise highlighted if the post-meal glucose target for that meal was met. The group of 7 icons 536 may be ordered in chronological order starting with Sunday, alternatively Monday. Each display or report 500, 520, 530 may further include an explanation of the display 504. For instance, the explanation of the display 504 may indicate that the report corresponds to how many times or days that the user reached their post-meal glucose target for that week. Each display or report 500, 520, 530 may further include a description of the glucose post meal target 506. For example, the glucose post meal target may be a glucose level below 180 mg/dL 2 hours after injection.

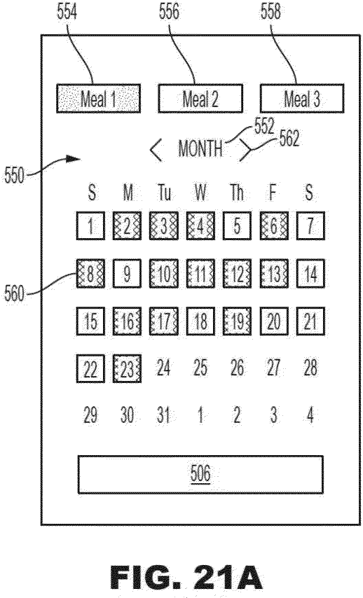

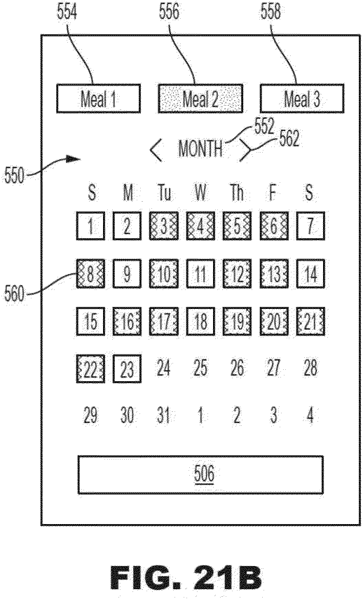

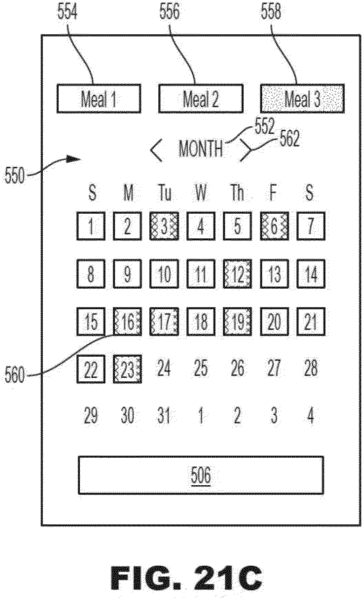

In another embodiment, as seen in FIGS. 21A-21C, a display or report 550 may include a metric or an indication of how times the user’s post-meal glucose target was reached during a given month for each meal (breakfast 554, lunch 556, and dinner 558). A GUI may display all of the days of the month 552, and days in which the glucose post meal target was met 560 may be highlighted for example, by shading or a different color number for the date. The user may toggle to view the results of different meals 554, 556, 558 by selecting the meal type. The user may also be able to toggle to different months by selecting the appropriate arrows 562. Each display or report 550 may further include a description of the glucose post meal target 506. For example, the glucose post meal target may be a glucose level below 180 mg/dL 2 hours after injection.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

Systems, devices, and methods for identifying and managing medication dosage patterns to assist in decisions for administration at, e.g., the time a meal is being consumed, are described. The application can include a new mealtime insulin dose-decisioning feature that is accessible through or in conjunction with an analyte monitoring application. Using retrospective analyte and medication dosing data only, the dosing pattern management application can display patterns in past dose administrations to facilitate easy and better dose decisions for diabetics on a regimen of multiple daily injections.

In many embodiments, a computer-implemented method for managing dosing patterns is described. The method includes the steps of receiving an analyte level of a user; determining an analyte range that contains the analyte level; referencing, by processing circuitry, a database to determine at least one medication dosage amount that was administered to the user when a measured analyte level was in the analyte range; and outputting at least a portion of the at least one medication dosage amount to an electronic display.

In some embodiments, the analyte level is a current analyte level of the user.

In some embodiments, the database comprises a plurality of medication dosage amounts paired with the measured analyte level of the user at or near a time of administration of each of the at least one medication dosage amount.

In some embodiments, outputting the at least a portion of the at least one medication dosage amount comprises displaying the at least a portion of the at least one medication dosage amount.

In some embodiments, the method further includes the step of analyzing, by processing circuitry, the at least one medication dosage amount to determine a smallest dose and a largest dose administered. In some embodiments, the method further includes the step of analyzing, by processing circuitry, the at least one medication dosage amount to determine a mode dose administered. In some embodiments, outputting the at least a portion of the at least one medication dosage amount comprises displaying the smallest dose, the largest dose, and the mode dose administered. In some embodiments, outputting the at least a portion of the at least one medication dosage amount comprises displaying a time or date when each of the smallest dose, the largest dose, and the mode dose were administered. In some embodiments, the at least a portion of the at least one medication dosage amount are divided into groups based on a time of day that each of the at least a portion of the plurality of medication dosage amounts was administered. In some embodiments, each of the at least a portion of the at least one medication dosage amount are divided into groups based on an association with a meal with which each of the at least a portion of the plurality of medication dosage amounts was administered. In some embodiments, the groups based on the association with the meal include breakfast, lunch, and dinner. In some embodiments, each of the at least a portion of the at least one medication dosage amount are divided into groups, and wherein each of the at least a portion of the at least one medication dosage amount for a single group may be displayed.

In some embodiments, the method further includes the step of analyzing, by processing circuitry, the at least a portion of the at least one medication dosage amount to determine if administration of each of the at least a portion of the at least one medication dosage amount resulted in a measured analyte level within a goal range after a period of time after administration of each of the at least a portion of the at least one medication dosage amount. In some embodiments, the period of time after administration is about two hours. In some embodiments, the goal range is selected from the group consisting of below about 180 mg/dL, below about 160 mg/dL, and below about 140 mg/dL. In some embodiments, an indication of a medication dosage amount of the at least a portion of the at least one medication dosage amount that resulted in a measured analyte level within the goal range is visibly distinguishable from an indication of a medication dosage amount of the at least a portion of the at least one medication dosage amount that did not result in a measured analyte level within the goal range. In some embodiments, the indication of a medication dosage amount of the at least a portion of the at least one medication dosage amount that resulted in a measured analyte level within the goal range is colored green.

In some embodiments, the method further includes the step of analyzing, by processing circuitry, the at least a portion of the at least one medication dosage amount to determine if administration of each of the at least a portion of the at least one medication dosage amount resulted in a measured analyte level below about 70 mg/dL about 4 hours after administration of each of the at least a portion of the at least one medication dosage amount. In some embodiments, an indication of a medication dosage amount of the at least a portion of the at least one medication dosage amount that resulted in a measured analyte level below about 70 mg/dL about 4 hours after administration is visibly distinguishable from an indication of a medication dosage amount of the at least a portion of the at least one medication dosage amount that did not result in a measured analyte level below about 70 mg/dL about 4 hours after administration. In some embodiments, the indication of a medication dosage amount of the at least a portion of the at least one medication dosage amount that resulted in a measured analyte level below about 70 mg/dL about 4 hours after administration includes an emoji.

In some embodiments, the analyte range is defined by the analyte level ± 25%.

In some embodiments, an upper and a lower boundary of the analyte range are selected from the group consisting of the analyte level ± 30%, the analyte level ± 25%, the analyte level ± 20%, the analyte level ± 15%, the analyte level ± 10%, and the analyte level ± 5%.

In some embodiments, the analyte level is a glucose level.

In some embodiments, the at least one medication dosage amount is at least one insulin dosage amount.

In some embodiments, an upper and a lower boundary of the analyte range are selected from the group consisting of the analyte level ± 50 mg/dL, the analyte level ± 40 mg/dL, the analyte level ± 30 mg/dL, the analyte level ± 25 mg/dL, the analyte level ± 20 mg/dL, and the analyte level ± 10 mg/dL.

In some embodiments, the at least one medication dosage amount is a plurality of medication dosage amounts, and the method further includes the steps of: analyzing, by processing circuitry, the plurality of medication dosage amounts to order the plurality of medication dosage amounts according to the amount of each dose; and forming first, second and third groups from the plurality of medication dosage amounts. In some embodiments, the first group includes a smallest medication dosage administered to the user, the second group includes a largest medication dosage administered to the user, and the third group includes a median medication dosage administered to the user. In some embodiments, the method further includes the steps of determining, by processing circuitry, a mode dose for each of the first, second, and third groups, and displaying the mode dose for each of the first, second, and third groups.

In some embodiments, the method further includes the step of rating, by processing circuitry, the at least one medication dosage amount. In some embodiments, each of the plurality of medication dosage amounts are rated according to a proximity of a measured analyte level to a goal range after a period of time after administration of each of the plurality of medication dosages. In some embodiments, the period of time is about 2 hours after administration. In some embodiments, the plurality of medication dosage amounts are rated with stars. In some embodiments, outputting the at least a portion of the at least one medication dosage amount comprises displaying the at least a portion of the at least one medication dosage amount and a rating associated with each of the at least a portion of the at least one medication dosage amount. In some embodiments, the at least a portion of the at least one medication dosage amount are displayed according to the rating associated with each of the at least a portion of the at least one medication dosage amount.

In some embodiments, the method further includes the step of associating, by processing circuitry, a tag with at least a second portion of the at least one medication dosage amount. In some embodiments, the tag comprises details of a food, emotion, or activity associated with the at least a portion of the plurality of medication dosages. In some embodiments, outputting the at least a portion of the at least one medication dosage amount comprises displaying the at least a portion of the at least one medication dosage amount and the tag associated with each of the at least a second portion of the at least one medication dosage amount.

In many embodiments, an electronic system configured to display past medication dosage information is described. The system includes processing circuitry; and a non-transitory memory comprising a plurality of instructions that, when executed, causes the processing circuitry to: receive an analyte level of a user; determine an analyte range that contains the analyte level; reference a database to determine at least one medication dosage amount that was administered to the user when a measured analyte level was in the analyte range; and output at least a portion of the at least one medication dosage amount to an electronic display.

In some embodiments, the database comprises a plurality of medication dosage amounts paired with the measured analyte level of the user at or near a time of administration of each of the at least one medication dosage amount.

In some embodiments, outputting the at least a portion of the at least one medication dosage amount comprises displaying the at least a portion of the at least one medication dosage amount.

In some embodiments, the plurality of instructions, when executed, further causes the processing circuitry to analyze the at least one medication dosage amount to determine a smallest dose, a largest dose, and a mode dose administered. In some embodiments, the plurality of instructions, when executed, further causes the processing circuitry to display the smallest dose, the largest dose, and the mode dose.

In some embodiments, the plurality of instructions, when executed, further causes the processing circuitry to divide the least a portion of the at least one medication dosage amount into groups based on an association with a meal with which each of the at least a portion of the at least one medication dosage amount was administered.

In some embodiments, an upper and a lower boundary of the analyte range are selected from the group consisting of the analyte level ± 30%, the analyte level ± 25%, the analyte level ± 20%, the analyte level ± 15%, the analyte level ± 10%, and the analyte level ± 5%.

In some embodiments, an upper and a lower boundary of the analyte range are selected from the group consisting of the analyte level ± 50 mg/dL, the analyte level ± 40 mg/dL, the analyte level ± 30 mg/dL, the analyte level ± 25 mg/dL, the analyte level ± 20 mg/dL, and the analyte level ± 10 mg/dL.

In some embodiments, the at least one medication dosage amount is a plurality of medication dosage amounts, wherein the plurality of instructions, when executed, further causes the processing circuitry to: analyze the plurality of medication dosage amounts to order the plurality of medication dosage amounts according to the amount of each dose; and form first, second and third groups from the plurality of medication dosage amounts. In some embodiments, the first group includes a smallest medication dosage administered to the user, the second group includes a largest medication dosage administered to the user, and the third group includes a median medication dosage administered to the user. In some embodiments, the plurality of instructions, when executed, further causes the processing circuitry to: determine a mode dose for each of the first, second, and third groups, and display the mode dose for each of the first, second, and third groups.

In some embodiments, the plurality of instructions, when executed, further causes the processing circuitry to rate the at least one medication dosage amount. In some embodiments, each of the plurality of medication dosages are rated according to a proximity of a measured analyte level to a goal range after a period of time after administration of each of the at least one medication dosage amount.

In some embodiments, the plurality of instructions, when executed, further causes the processing circuitry to associate a tag with at least a second portion of the at least one medication dosage amount. In some embodiments, the tag comprises details of a food, emotion, or activity associated with the at least a portion of the plurality of medication dosages.

In many embodiments, a computer-implemented method for identifying and managing dosing patterns is provided. The method can include: receiving an analyte level of a user; determining an analyte range that contains the analyte level; referencing, by processing circuitry, a database to determine a plurality of medication dosage amounts that were administered to the user when a measured analyte level was in the analyte range; and outputting at least a portion of the plurality of medication dosage amounts to an electronic display.

In some embodiments, the analyte level is a current analyte level of the user.

In some embodiments, the database comprises a plurality of medication dosage amounts paired with the measured analyte level of the user at or near a time of administration of each of the plurality of medication dosage amounts.

In some embodiments, outputting the at least a portion of the plurality of medication dosage amounts comprises displaying the at least a portion of the plurality of medication dosage amounts.

In some embodiments, the method further includes a step of analyzing, by processing circuitry, the plurality of medication dosage amounts to determine a smallest dose and a largest dose administered. In some embodiments, the method further includes the step of analyzing, by processing circuitry, the plurality of medication dosage amounts to determine a mode dose administered. In some embodiments, outputting the at least a portion of the plurality of medication dosage amounts comprises displaying the smallest dose, the largest dose, and the mode dose administered. In some embodiments, outputting the at least a portion of the plurality of medication dosage amounts comprises displaying a time or date when each of the smallest dose, the largest dose, and the mode dose were administered. In some embodiments, at least a portion of the plurality of medication dosage amounts are divided into groups based on a time of day that each of the at least a portion of the plurality of medication dosage amounts was administered. In some embodiments, each of the at least a portion of the plurality of medication dosage amounts are divided into groups based on an association with a meal with which each of the at least a portion of the plurality of medication dosage amounts was administered. In some embodiments, the groups based on the association with the meal include breakfast, lunch, and dinner. In some embodiments, each of the at least a portion of the plurality of doses are divided into groups, and wherein each of the at least a portion of the plurality of doses for a single group may be displayed.

In some embodiments, the method further includes the step of analyzing, by processing circuitry, the at least a portion of the plurality of medication dosage amounts to determine if administration of each of the at least a portion of the plurality of medication dosage amounts resulted in a measured analyte level within a goal range after a period of time after administration of each of the at least a portion of the plurality of medication dosages. In some embodiments, the period of time after administration is about two hours. In some embodiments, the goal range is selected from the group consisting of below about 180 mg/dL, below about 160 mg/dL, and below about 140 mg/dL. In some embodiments, an indication of a medication dosage amount of the at least a portion of the plurality of medication dosage amounts that resulted in a measured analyte level within the goal range is visibly distinguishable from an indication of a medication dosage amount of the at least a portion of the plurality of medication dosage amounts that did not result in a measured analyte level within the goal range. In some embodiments, the indication of a medication dosage amount of the at least a portion of the plurality of medication dosage amounts that resulted in a measured analyte level within the goal range is colored green.

In some embodiments, the method further includes the step of analyzing, by processing circuitry, the at least a portion of the plurality of medication dosage amounts to determine if administration of each of the at least a portion of the plurality of medication dosage amounts resulted in a measured analyte level below about 70 mg/dL about 4 hours after administration of each of the at least a portion of the plurality of medication dosage amounts. In some embodiments, an indication of a medication dosage amount of the at least a portion of the plurality of medication dosage amounts that resulted in a measured analyte level below about 70 mg/dL about 4 hours after administration is visibly distinguishable from an indication of a medication dosage amount of the at least a portion of the plurality of medication dosage amounts that did not result in a measured analyte level below about 70 mg/dL about 4 hours after administration. In some embodiments, the indication of a medication dosage amount of the at least a portion of the plurality of medication dosage amounts that resulted in a measured analyte level below about 70 mg/dL about 4 hours after administration includes an emoji.

In some embodiments, the analyte range is defined by the analyte level ± 25%.

In some embodiments, an upper and a lower boundary of the analyte range are selected from the group consisting of the analyte level ± 30%, the analyte level ± 25%, the analyte level ± 20%, the analyte level ± 15%, the analyte level ± 10%, and the analyte level ± 5%.

In some embodiments, the analyte level is a glucose level.

In some embodiments, the plurality of medication dosage amounts are a plurality of insulin dosage amounts.

In some embodiments, an upper and a lower boundary of the analyte range are selected from the group consisting of the analyte level ± 50 mg/dL, the analyte level ± 40 mg/dL, the analyte level ± 30 mg/dL, the analyte level ± 25 mg/dL, the analyte level ± 20 mg/dL, and the analyte level ± 10 mg/dL.

In some embodiments, the method further includes the step of analyzing, by processing circuitry, the plurality of medication dosage amounts to order the plurality of medication dosage amounts according to the amount of each dose; and forming first, second and third groups from the plurality of medication dosage amounts. In some embodiments, the first group includes a smallest medication dosage administered to the user, the second group includes a largest medication dosage administered to the user, and the third group includes a median medication dosage administered to the user. In some embodiments, the method further includes the steps of determining, by processing circuitry, a mode dose for each of the first, second, and third groups, and displaying the mode dose for each of the first, second, and third groups.

In some embodiments, the method further includes the step of rating, by processing circuitry, the plurality of medication dosages. In some embodiments, each of the plurality of medication dosages are rated according to a proximity of a measured analyte level to a goal range after a period of time after administration of each of the plurality of medication dosages. In some embodiments, the period of time is about 2 hours after administration. In some embodiments, the plurality of medication dosages are rated with stars. In some embodiments, outputting the at least a portion of the plurality of medication dosages comprises displaying the at least a portion of the plurality of medication dosages and a rating associated with each of the at least a portion of the plurality of medication dosages. In some embodiments, at least a portion of the plurality of medication dosages are displayed according to the rating associated with each of the at least a portion of the plurality of medication dosages.

In some embodiments, the method further includes the step of associating, by processing circuitry, a tag with at least a second portion of the plurality of medication dosages. In some embodiments, the tag comprises details of a food, emotion, or activity associated with the at least a portion of the plurality of medication dosages. In some embodiments, outputting the plurality of medication dosages comprises displaying the at least a portion of the plurality of medication dosages and the tag associated with each of the at least a second portion of the plurality of medication dosages.

In many embodiments, an electronic system configured to display past medication dosage information, the system comprising: processing circuitry; and a non-transitory memory comprising a plurality of instructions that, when executed, causes the processing circuitry to: receive an analyte level of a user; determine an analyte range that contains the analyte level; reference a database to determine a plurality of medication dosage amounts that were administered to the user when a measured analyte level was in the analyte range; and output at least a portion of the plurality of medication dosage amounts to an electronic display.

In some embodiments, the database comprises a plurality of medication dosage amounts paired with the measured analyte level of the user at or near a time of administration of each of the plurality of medication dosage amounts.

In some embodiments, outputting the at least a portion of the plurality of medication dosage amounts comprises displaying the at least a portion of the plurality of medication dosage amounts.

In some embodiments, the plurality of instructions, when executed, further causes the processing circuitry to analyze the plurality of medication dosage amounts to determine a smallest dose, a largest dose, and a mode dose administered. In some embodiments, the plurality of instructions, when executed, further causes the processing circuitry to display the smallest dose, the largest dose, and the mode dose.

In some embodiments, the plurality of instructions, when executed, further causes the processing circuitry to divide the least a portion of the plurality of medication dosage amounts into groups based on an association with a meal with which each of the at least a portion of the plurality of medication dosage amounts was administered.

In some embodiments, an upper and a lower boundary of the analyte range are selected from the group consisting of the analyte level ± 30%, the analyte level ± 25%, the analyte level ± 20%, the analyte level ± 15%, the analyte level ± 10%, and the analyte level ± 5%.

In some embodiments, an upper and a lower boundary of the analyte range are selected from the group consisting of the analyte level ± 50 mg/dL, the analyte level ± 40 mg/dL, the analyte level ± 30 mg/dL, the analyte level ± 25 mg/dL, the analyte level ± 20 mg/dL, and the analyte level ± 10 mg/dL.

In some embodiments, the plurality of instructions, when executed, further causes the processing circuitry to: analyze the plurality of medication dosage amounts to order the plurality of medication dosage amounts according to the amount of each dose; and form first, second and third groups from the plurality of medication dosage amounts. In some embodiments, the first group includes a smallest medication dosage administered to the user, the second group includes a largest medication dosage administered to the user, and the third group includes a median medication dosage administered to the user. In some embodiments, the plurality of instructions, when executed, further causes the processing circuitry to: determine a mode dose for each of the first, second, and third groups, and display the mode dose for each of the first, second, and third groups.

In some embodiments, the plurality of instructions, when executed, further causes the processing circuitry to rate the plurality of medication dosages. In some embodiments, each of the plurality of medication dosages are rated according to a proximity of a measured analyte level to a goal range after a period of time after administration of each of the plurality of medication dosages.

In some embodiments, the plurality of instructions, when executed, further causes the processing circuitry to associate a tag with at least a second portion of the plurality of medication dosages. In some embodiments, the tag comprises details of a food, emotion, or activity associated with the at least a portion of the plurality of medication dosages.

In many embodiments, a computer-implemented method for assisting in diabetes management is described. The method includes the steps of receiving data comprising an analyte level of a user within a period of time after an amount of insulin has been administered for each day of a plurality of days in a time period; determining a subset of the data comprising a number of analyte levels for each of the plurality of days that satisfy a glucose target condition; and displaying a plurality of icons comprising an icon for each day of the plurality of days in the time period, wherein a number of icons of the plurality of icons equal to the number of corresponding analyte levels in the subset are visually distinct from a remaining number of icons of the plurality of icons that are not in the subset.

In some embodiments, the time period is one week.

In some embodiments, the time period is one month.

In some embodiments, the glucose target condition comprises an analyte level below an upper glucose threshold. In some embodiments, the upper glucose threshold is between about 170 mg/dL and about 190 mg/dL.

In some embodiments, the data further comprises the amount of insulin administered for each day of the plurality of days in the time period.

In some embodiments, the number of icons of the plurality of icons equal to the number of corresponding analyte levels in the subset are a different color than the remaining number of icons of the plurality of icons that are not in the subset.

In some embodiments, the number of icons of the plurality of icons equal to the number of corresponding analyte levels in the subset are bolder than the remaining number of icons of the plurality of icons that are not in the subset.

In some embodiments, the number of icons of the plurality of icons equal to the number of corresponding analyte levels in the subset are emojis and the remaining number of icons of the plurality of icons that are not in the subset are not emojis.

In some embodiments, the number of icons of the plurality of icons equal to the number of corresponding analyte levels in the subset are displayed next to each other.

In some embodiments, the number of icons of the plurality of icons equal to the number of corresponding analyte levels in the subset are displayed in a chronological order according to a corresponding day.

In some embodiments, the plurality of icons displayed are further divided according to a meal associated with each analyte level of the user within the period of time after the amount of insulin has been administered for each day of a plurality of days in the time period.

In some embodiments, the plurality of icons displayed are further divided into icons corresponding to breakfast, lunch, and dinner.

In many embodiments, an electronic system configured to display information related to diabetes management is described. The system includes processing circuitry; and a non-transitory memory comprising a plurality of instructions that, when executed, causes the processing circuitry to: receive an analyte level of a user within a period of time after an amount of insulin has been administered for each day of a plurality of days in a time period; determine a subset of the data comprising a number of analyte levels for each of the plurality of days that satisfy a glucose target condition; and display a plurality of icons comprising an icon for each day of the plurality of days in the time period, wherein a number of icons of the plurality of icons equal to the number of corresponding analyte levels in the subset are visually distinct from a remaining number of icons of the plurality of icons that are not in the subset.

In some embodiments, the time period is one week.

In some embodiments, the time period is one month.

In some embodiments, the glucose target condition comprises an analyte level below an upper glucose threshold. In some embodiments, the upper glucose threshold is between about 170 mg/dL and about 190 mg/dL.

In some embodiments, the data further comprises the amount of insulin administered for each day of the plurality of days in the time period.

In some embodiments, the number of icons of the plurality of icons equal to the number of corresponding analyte levels in the subset are a different color than the remaining number of icons of the plurality of icons that are not in the subset.

In some embodiments, the number of icons of the plurality of icons equal to the number of corresponding analyte levels in the subset are bolder than the remaining number of icons of the plurality of icons that are not in the subset.

In some embodiments, the number of icons of the plurality of icons equal to the number of corresponding analyte levels in the subset are emojis and the remaining number of icons of the plurality of icons that are not in the subset are not emojis.

In some embodiments, the number of icons of the plurality of icons equal to the number of corresponding analyte levels in the subset are displayed next to each other.

In some embodiments, the number of icons of the plurality of icons equal to the number of corresponding analyte levels in the subset are displayed in a chronological order according to a corresponding day.

In some embodiments, the plurality of icons displayed are further divided according to a meal associated with each analyte level of the user within the period of time after the amount of insulin has been administered for each day of a plurality of days in the time period.

In some embodiments, the plurality of icons displayed are further divided into icons corresponding to breakfast, lunch, and dinner.

Embodiments of the method can further include analyzing, by processing circuitry, the plurality of medication dosage amounts to determine a smallest dose, a largest dose, and/or a mode dose administered. In some embodiments, the method can further include analyzing, by processing circuitry, the at least one medication dosage amount to determine a smallest dose, a largest dose, and a mode dose administered. In some embodiments, the method can further include dividing the at least a portion of the plurality of medication dosage amounts into groups based on a time of day that each of the at least a portion of the plurality of medication dosage amounts was administered.

Embodiments of the method can further include analyzing, by processing circuitry, the at least a portion of the plurality of medication dosage amounts to determine if administration of each of the at least a portion of the plurality of medication dosage amounts resulted in a measured analyte level within a goal range after a period of time after administration of each of the at least a portion of the plurality of medication dosages. Embodiments of the method can further include analyzing, by processing circuitry, the at least a portion of the plurality of medication dosage amounts to determine if administration of each of the at least a portion of the plurality of medication dosage amounts resulted in a measured analyte level below about 70 mg/dL about 4 hours after administration of each of the at least a portion of the plurality of medication dosage amounts.

In certain example embodiments, the database comprises a plurality of medication dosage amounts paired with the measured analyte level of the user at or near a time of administration of each of the plurality of medication dosage amounts. In certain embodiments, boundaries of the analyte range can change or vary over time as more data is collected.

In certain example embodiments, outputting the at least a portion of the plurality of medication dosage amounts comprises displaying the smallest dose, the largest dose, and the mode dose administered. In certain example embodiments, outputting the at least a portion of the plurality of medication dosage amounts comprises displaying a day, time, or date when each of the smallest dose, the largest dose, and the mode dose were administered. In certain example embodiments, at least a portion of the plurality of medication dosage amounts are divided into groups based on a time of day that each of the at least a portion of the plurality of medication dosage amounts was administered. In certain example embodiments, at least a portion of the plurality of medication dosage amounts are divided into groups based on an association with a meal with which each of the at least a portion of the plurality of medication dosage amounts was administered. In certain example embodiments, the groups based on the association with the meal include breakfast, lunch, and dinner.

Embodiments of the method can further include analyzing, by processing circuitry, the plurality of medication dosage amounts to order the plurality of medication dosage amounts according to the amount of each dose; and forming first, second and third groups from the plurality of medication dosage amounts. Embodiments of the method can further include determining, by processing circuitry, a mode dose for each of the first, second, and third groups, and displaying the mode dose for each of the first, second, and third groups.

In certain example embodiments, the first group includes a smallest medication dosage administered to the user, the second group includes a largest medication dosage administered to the user, and the third group includes a median medication dosage administered to the user. In certain example embodiments, a mode dose for each of the first, second, and third groups are displayed.

Embodiments of the method can further include the step of rating, by processing circuitry, the plurality of medication dosages. In certain embodiments, each of the plurality of medication dosages are rated according to a proximity of a measured analyte level to a goal range after a period of time (e.g., about 2 hours) after administration of each of the plurality of medication dosages. In certain embodiments, the plurality of medication dosages are rated with stars. In certain embodiments, outputting the at least a portion of the plurality of medication dosages comprises displaying the at least a portion of the plurality of medication dosages and a rating associated with each of the at least a portion of the plurality of medication dosages. In certain embodiments, the at least a portion of the plurality of medication dosages are displayed according to the rating associated with each of the at least a portion of the plurality of medication dosages.

Embodiments of the method can further include the step of associating, by processing circuitry, a tag with at least a second portion of the plurality of medication dosages. In certain embodiments, the tag comprises details of a food, emotion, or activity associated with the at least a portion of the plurality of medication dosages. In certain embodiments, outputting the plurality of medication dosages comprises displaying the at least a portion of the plurality of medication dosages and the tag associated with each of the at least a second portion of the plurality of medication dosages.

In certain example embodiments, an electronic system configured to display past medication dosage information is provided that can include processing circuitry; and a non-transitory memory comprising a plurality of instructions that, when executed, causes the processing circuitry to: receive an analyte level of a user; determine an analyte range that contains the analyte level; reference a database to determine a plurality of medication dosage amounts that were administered to the user when a measured analyte level was in the analyte range; and output at least a portion of the plurality of medication dosage amounts to an electronic display.

In certain example embodiments, the database comprises a plurality of medication dosage amounts paired with the measured analyte level of the user at or near a time of administration of each of the plurality of medication dosage amounts. In certain example embodiments, outputting the at least a portion of the plurality of medication dosage amounts comprises displaying the at least a portion of the plurality of medication dosage amounts. In certain example embodiments, the plurality of instructions, when executed, further causes the processing circuitry to analyze the plurality of medication dosage amounts to determine a smallest dose, a largest dose, and a mode dose administered. In certain example embodiments, the plurality of instructions, when executed, further causes the processing circuitry to display the smallest dose, the largest dose, and the mode dose. In certain example embodiments, the plurality of instructions, when executed, further causes the processing circuitry to divide the least a portion of the plurality of medication dosage amounts into groups based on an association with a meal with which each of the at least a portion of the plurality of medication dosage amounts was administered.

In certain example embodiments, the analyte range is defined by the analyte level ± 25%. In certain example embodiments, an upper and a lower boundary of the analyte range are selected from the group consisting of the analyte level ± 30%, the analyte level ± 25%, the analyte level ± 20%, the analyte level ± 15%, the analyte level ± 10%, and the analyte level ± 5%. In certain example embodiments, an upper and a lower boundary of the analyte range are selected from the group consisting of the analyte level ± 50 mg/dL, the analyte level ± 40 mg/dL, the analyte level ± 30 mg/dL, the analyte level ± 25 mg/dL, the analyte level ± 20 mg/dL, and the analyte level ± 10 mg/dL.

In certain example embodiments, the plurality of instructions, when executed, further causes the processing circuitry to: analyze the plurality of medication dosage amounts to order the plurality of medication dosage amounts according to the amount of each dose; and form first, second and third groups from the plurality of medication dosage amounts. In certain example embodiments, the plurality of instructions, when executed, further causes the processing circuitry to: determine a mode dose for each of the first, second, and third groups, and display the mode dose for each of the first, second, and third groups. In certain example embodiments, the first group includes a smallest medication dosage administered to the user, the second group includes a largest medication dosage administered to the user, and the third group includes a median medication dosage administered to the user.

In certain example embodiments, the plurality of instructions, when executed, further causes the processing circuitry to rate the plurality of medication dosages. In certain example embodiments, each of the plurality of medication dosages are rated according to a proximity of a measured analyte level to a goal range after a period of time after administration of each of the plurality of medication dosages.

In certain example embodiments, the plurality of instructions, when executed, further causes the processing circuitry to associate a tag with at least a second portion of the plurality of medication dosages. In certain example embodiments, the tag comprises details of a food, emotion, or activity associated with the at least a portion of the plurality of medication dosages.

The improvements to the GUIs in the various aspects described and claimed herein produce a technical effect at least in that they assist the user of the device to operate the device more accurately, more efficiently and more safely. It will be appreciated that the information that is provided to the user on the GUI, the order in which that information is provided and the clarity with which that information is structured can have a significant effect on the way the user interacts with the system and the way the system operates. The GUI therefore guides the user in the technical task of operating the system to take the necessary readings and/or obtain information accurately and efficiently.

All features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

As used herein and in the appended claims, the singular forms “a,” “an,” and “the” include plural referents unless the context clearly dictates otherwise.

Aspects of the invention are set out in the independent claims and preferred features are set out in the dependent claims. The preferred features of the dependent claims may be provided in combination in a single embodiment and preferred features of one aspect may be provided in conjunction with other aspects.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1-37. (canceled)

38. An electronic system configured to display past medication dosage information, the system comprising:
processing circuitry; and
a non-transitory memory comprising a plurality of instructions that, when executed, causes the processing circuitry to:
receive an analyte level of a user;
determine an analyte range that contains the analyte level;
reference a database to determine at least one medication dosage amount that was administered to the user when a measured analyte level was in the analyte range; and
output at least a portion of the at least one medication dosage amount to an electronic display.

39. The system of claim 38, wherein the database comprises a plurality of medication dosage amounts paired with the measured analyte level of the user at or near a time of administration of each of the at least one medication dosage amount.

40. The system of claim 38, wherein outputting the at least a portion of the at least one medication dosage amount comprises displaying the at least a portion of the at least one medication dosage amount.

41. The system of claim 38, wherein the plurality of instructions, when executed, further causes the processing circuitry to analyze the at least one medication dosage amount to determine a smallest dose, a largest dose, and a mode dose administered.

42. The system of claim 41, wherein the plurality of instructions, when executed, further causes the processing circuitry to display the smallest dose, the largest dose, and the mode dose.

43. The system of claim 38, wherein the plurality of instructions, when executed, further causes the processing circuitry to divide the least a portion of the at least one medication dosage amount into groups based on an association with a meal with which each of the at least a portion of the at least one medication dosage amount was administered.

44. The system of claim 38, wherein an upper and a lower boundary of the analyte range are selected from the group consisting of the analyte level ± 30%, the analyte level ± 25%, the analyte level ± 20%, the analyte level ± 15%, the analyte level ± 10%, and the analyte level ± 5%.

45. The system of claim 38, wherein an upper and a lower boundary of the analyte range are selected from the group consisting of the analyte level ± 50 mg/dL, the analyte level ± 40 mg/dL, the analyte level ± 30 mg/dL, the analyte level ± 25 mg/dL, the analyte level ± 20 mg/dL, and the analyte level ±10 mg/dL.

46. The system of claim 38, wherein the at least one medication dosage amount is a plurality of medication dosage amounts, wherein the plurality of instructions, when executed, further causes the processing circuitry to:
analyze the plurality of medication dosage amounts to order the plurality of medication dosage amounts according to the amount of each dose; and
form first, second and third groups from the plurality of medication dosage amounts.

47. The system of claim 46, wherein the first group includes a smallest medication dosage administered to the user, the second group includes a largest medication dosage administered to the user, and the third group includes a median medication dosage administered to the user.

48. The system of claim 46, wherein the plurality of instructions, when executed, further causes the processing circuitry to:
determine a mode dose for each of the first, second, and third groups, and
display the mode dose for each of the first, second, and third groups.

49. The system of claim 38, wherein the plurality of instructions, when executed, further causes the processing circuitry to rate the at least one medication dosage amount.

50. The system of claim 49, wherein each of the plurality of medication dosages are rated according to a proximity of a measured analyte level to a goal range after a period of time after administration of each of the at least one medication dosage amount.

51. The system of claim 38, wherein the plurality of instructions, when executed, further causes the processing circuitry to associate a tag with at least a second portion of the at least one medication dosage amount.

52. The system of claim 51, wherein the tag comprises details of a food, emotion, or activity associated with the at least a portion of the plurality of medication dosages.

53-78. (canceled)
